(19) United States
(12) Patent Application Publication
(10) Pub. No.: US 2023/0126411 A1
(43) Pub. Date: Apr. 27, 2023

(54) DEUTERIUM MAGNETIC RESONANCE IMAGING

(71) Applicant: University of Florida Research Foundation, Inc., Gainesville, FL (US)

(72) Inventors: Patrick DONABEDIAN, Gainesville, FL (US); Rohit MAHAR, Gainesville, FL (US); Matthew E. MERRITT, Gainesville, FL (US)

(21) Appl. No.: 17/906,508

(22) PCT Filed: Jan. 7, 2021
(86) PCT No.: PCT/US2021/012400

§ 371 Date: Sep. 16, 2022

Related U.S. Application Data
(60) Provisional application No. 62/992,726, filed on Mar. 20, 2020.
(60) Provisional application No. 63/004,145, filed on Apr. 2, 2020.
(60) Provisional application No. 63/120,996, filed on Dec. 3, 2020.

(51) Int. Cl. A61B 5/055; A61B 5/145; G01R 33/56

(52) U.S. Cl. CPC A61B 5/055; A61B 5/14532; G01R 33/5601

(57) ABSTRACT

Disclosed herein are methods for imaging a tissue in a subject that involves administering to the subject a composition comprising deuterium-labeled glycolytic or fatty acid substrate and imaging the subject with deuterium magnetic resonance imaging (DMI) to detect hydrogen-deuterium oxide (HDO) in tissues of the subject. The disclosed methods can be used to detect changes in metabolic activity in a tissue. The disclosed methods can also be used to detect cancers.

### CROSS-REFERENCE TO RELATED APPLICATIONS

This application claims benefit of U.S. Provisional Application No. 62/992,726, filed Mar. 20, 2020, U.S. Provisional Application No. 63/004,145, filed Apr. 2, 2020, and U.S. Provisional Application No. 63/120,996, filed Dec. 3, 2020, which are hereby incorporated herein by reference in their entireties.

### BACKGROUND

Central carbon metabolism plays an important role in the origin or progression of many diseases, including cancers, neurodegenerative diseases, diabetes, and nonalcoholic fatty liver disease (NAFLD) (Zilberter, Y. & Zilberter, M. J Neurosci Res. 2017 95:2217-2235; Pavlova, N. N. & Thompson, C. B. Cell Metabolism 2016 23:27-47; Samuel, V T. & Shulman, G I. Cell Metabolism 2018 27:22-41). Metabolic reprogramming is one of the key features of cancer cell metabolism. Many cancer cells consume tremendous amounts of glucose and generate ATP via glycolysis even under normoxic conditions (the Warburg effect) (Bensinger, S J. & Christofk, H R. Semin Cell Dev Biol. 2012 23:352-361; Warburg, O. Science 1956 124:269-270; Warburg, O. The J Cancer Res 1925 9:148-163). Many theories have been promulgated to explain this observation, ranging from impaired mitochondrial function as the primary causative agent of cancer (Warburg, O. Science 1956 123:309-314), to a trade-off of inefficient energy metabolism for faster incorporation of carbon into biomass (Vander Heiden, M G., et al. Science 2009 324:1029-1033), to a double selective advantage of hypoxia resistance and generation of local acidosis (Gatenby, R A. & Gillies, R J. Nat Rev Cancer. 2004 4:891-899). Molecular imaging techniques capable of identifying such differences have become very important for cancer diagnosis, disease progression, and treatment.

The kinetics of lactate and HDO (or ²HOH) production can serve as a quick and reliable metric for cancer diagnosis or staging. Glucose tracers have been widely utilized for accessing the metabolic conversion of glucose to lactate predominantly via glycolysis. Metabolic imaging with radioactive agents, which relies on physiologic hallmarks unique to cancer (Hanahan, D. & Weinberg, R A. Cell 2011 144:646-674), may be more sensitive but its widespread use is limited. In the clinic, the glucose analog [¹⁸F]2-fluoro-2-deoxy-glucose (¹⁸FDG) is used for positron emission tomography (PET) high-resolution maps of glucose uptake, but cannot report differences in metabolism and is associated with doses of ionizing radiation that carry an unknown, but real, risk of secondary cancer (Linet, M. S. et al. C A Cancer J Clin. 2012 62(2):75-100; Kaushik, A. et al. Indian J Med Res. 2015 142(6):721-31). These tests are therefore limited to patients with known or suspected malignancy. ¹⁸FDG is radioactive, which is also a limitation when repetitive scanning is required for evaluating disease progression (Kim, M M., et al. Nat Rev Clin Oncol 2016 13:725-739). CEST MRI was developed to overcome some of the limitations of PET for glucose imaging. CEST detects glucose uptake indirectly by saturation transfer from the exchangeable protons of glucose to water, which enhances sensitivity relative to direct detection, but the technique is hampered by water exchange rates at physiological pH (Walker-Samuel, S. et al. Nat Med 2013 19:1067-1072; van Zijl, P C., et al. Proc Natl Acad Sci USA. 2007 104(11):4359-64). Dissolution dynamic nuclear polarization (dDNP) was developed to enhance signal-to-noise ratio (SNR) in ¹³C MR via polarization transfer from an electron source to nuclei, resulting in >10,000 times signal enhancement in the MRI signal (Ardenkjaer-Larsen, J H. et al. Proc Natl Acad Sci USA. 2003 100:10158-10163; Kurhanewicz, J. et al. Neoplasia 2011 13:81-97). However, dDNP is limited to a small set of molecules having long relaxation times to measure the kinetics before polarized signal is lost. Many important metabolites, i.e. glucose, have short relaxation times, and are difficult to image using dDNP methods (Comment A, et al. Biochemistry. 2014 53(47):7333-57). Lactate pool size convolves with estimates of lactate production when estimated with hyperpolarized pyruvate, which in a strict biochemical sense is not measuring glycolysis. Analysis of lactate isotopomers has been carried out using ¹³C labeled glucose and spin editing, but the ¹³C enrichment is not easily determined in some cases due to overlap with fatty acid (FA) resonances (Jucker, B M., et al. J. Biol. Chem. 1997 272:10464-10473). Due to the multiplicities from nuclear spin-spin couplings of the ¹H-¹³C in lactate ¹³C-isotopomers, ¹H-NMR spectra become complex. Direct ¹³C acquisition provides a straightforward method for isotopomer analysis but requires long data acquisition time (Lloyd, S G., et al. Magn. Reson. Med. 2004 51:1279-1282). Therefore, improved methods for in vivo detection of glucose utilization by tumor cells are needed.

Non-alcoholic fatty liver disease (NAFLD) and non-alcoholic steatohepatitis (NASH) are public health risks that are rapidly growing (Younossi, Z, et al. Nature reviews Gastroenterology & Hepatology 2018 15(1):11). NASH, the more medically serious condition, is predicted to become the number one cause of liver transplant in the next decade (McPherson, S, et al. Journal of hepatology 2015 62(5):1148-1155). Liver biopsy has remained the primary criterion standard or “gold standard” in the evaluation of the etiology and extent of disease of the liver such as NAFLD and NASH. Percutaneous liver biopsy is the preferred method to determine NAFLD and to differentiate NASH from NAFLD (Nalbantoglu, I, et al. World journal of gastroenterology 2014 20(27):9026). Other biopsy methods are typically even more invasive and include transvenous and laparoscopic liver biopsy. Although liver biopsies are generally regarded as safe, they bare risks that are potentially lethal. Almost two thirds of complications of liver biopsy occur within two hours. Approximately 2% of patients undergoing liver biopsy require hospitalization for the management of an adverse event. Significant bleeding after a liver biopsy occurs in approximately 1% of patients who are biopsied (Perrault, J, et al. Gastroenterology 1978 74(1):103-106). If bleeding persists, a blood transfusion may be needed. Surgery or angiography, where the bleeding site is identified and treated, may be required if the bleeding is severe or does not stop on its own. Intraperitoneal hemorrhage is the most serious consequence of bleeding. Liver biopsy is also handicapped by sampling error, which can result in misdiagnosis (Ratziu, V, et al. Gastroenterology 2005 128(7):1898-1906). Thus, a robust minimally invasive test to reliably and efficiently diagnose NASH and differentiate the inflammatory indication NASH from the less harmful NAFLD, and which also provides an assessment of the entire liver, is needed.

### SUMMARY

As disclosed herein, deuterium (²H) magnetic resonance imaging (DMI), a new method for assessing metabolic flux in functioning tissues, cell culture, and in vivo, provides the requisite chemical, spatial and temporal resolution needed for detecting glucose metabolism in tissues and cancers (De Feyter, H M. et al. et al. Sci Adv 2018 4:eaat7314). Nonradioactive ²H-labeled substrates can be utilized to observe the downstream metabolites, due to very low natural abundance of ²H (0.0115%), requiring no natural abundance correction for analysis. Administration of deuterium-labeled substrates at tracer levels (<200 mg/kg ²H) is well established as safe (Klein, D. Stable Isotopes 1986 378-382; Jones, P J H. & Leatherdale, S T. Clin Sci (Lond). 1991 80(4):277-80; Koletzko, B., et al. Eur J Pediatr. 1997 156 Suppl 1:S12-7). In vivo ²H MR was investigated in the late 1980's (Ackerman, J J H., et al. Proc Natl Acad Sci USA. 1987 84:4099-4102; Ewy, C S., et al. Magnetic Resonance in Medicine 1988 8:35-44; Ewy, C S., et al. Magnetic Resonance Imaging 1986 4:407-411; Müller, S. & Seelig, J. Journal of Magnetic Resonance 1969 72:456-466), but has recently enjoyed a resurgence due to improved technology and the accumulation of decades of basic research on cancer metabolic phenotypes (Tee, S. & Keshari, K R. The Cancer Journal 2015 21:165-173). Previous studies using ²H MRI to image cancer metabolism relied on differential production of TCA cycle intermediates and lactate (De Feyter, H M. et al. Sci Adv. 2018 4(8):eaat7314). Deuterated tracers are inexpensive and straightforward to administer, but ²H MR sensitivity is low. Indeed, the experiment is only tractable because the quadrupole of the ²H nucleus makes its T₁ short, facilitating very short recycle time (Tr) To cope with low sensitivity of ²H-NMR experiment for less abundant metabolites, more sensitive experiments and methodology were designed.

As disclosed herein, enrichment of ²HOH (i.e. HDO production) after administration of deuterium-labeled glucose will provide a sensitive marker of glucose utilization in tissues and tumor cells in vivo, with a possibility for a simplified imaging strategy. Likewise, administration of deuterium-labeled fatty acid will provide a sensitive marker of fatty acid (FA) beta-oxidation. All living tissues are oxidative in nature. Therefore, one could expect both glucose and any fatty acid to produce HDO by the disclosed methods in any functioning tissue including the brain, heart, muscle, skin, liver, pancreas, spleen, kidney, stomach, gut, or lungs. HDO generated from oxidation of glucose or a fatty acid could be used to detect either an increment or decrement of metabolic activity in any organ or tissue. These changes could be derived from inborn errors in metabolism.

Therefore, disclosed herein is a method for imaging a tissue in a subject, comprising administering to the subject a composition comprising deuterium-labeled glycolytic of FA substrate; and imaging the subject with deuterium magnetic resonance imaging (DMI) to detect hydrogen-deuterium oxide (HDO) in tissues of the subject, wherein detection of HDO in a location within the tissue of the subject is an indication of glucose utilization or FA oxidation in the tissue of the subject, respectively.

This method could therefore be used to detect any disease in the brain that results in changing metabolism as well as traumatic brain injury. This method could also be used to detect normal brain activation, in analogy to functional MRI (fMRI). As another example, HDO generation could be used to detect any increment/decrement in heart function, due to exercise, or due to heart hypertrophy or failure. Heart dysfunction would be detected by HDO generation from glucose and/or a fatty acid whether it was caused by over-pressure heart failure or via diabetic cardiomyopathy. HDO generation could be used to detect any increment/decrement in muscle function or in exercise physiology. Any disease of the muscle that changed its metabolism would be sensitive to HDO generation from glucose or a FA. HDO generated from glucose metabolism in the pancreas could also be used to detect glucose stimulated insulin secretion. HDO generated from glucose or FA metabolism could also be used to detect kidney function.

Lactate production from glucose is a hallmark of many cancers. The disclosed methods can therefore also be used to detect cancer based on glycolytic flux due to this Warburg Effect. If the glycolytic product is enriched with deuterium, HDO is produced along with the lactate, which can then be detected. The disclosed methods involve first administering to the subject a deuterium-labeled glycolytic substrate (e.g. glucose), which will be metabolized into lactate and hydrogen-deuterium oxide (HDO) in cancers. In addition, deuterium-induced perturbations of ¹H-NMR chemical shifts (isotope shift) allows resolution of the lactate isotopomers.

Therefore, disclosed herein is a method for detecting a tumor in a subject, that involves administering to the subject a composition comprising deuterium-labeled glycolytic substrate; and imaging the subject with deuterium magnetic resonance imaging (DMI) to detect hydrogen-deuterium oxide (HDO) in tissues of the subject, wherein detection of elevated HDO in a location within the tissue of the subject is an indication of the presence of cancerous tissue in the subject.

In some embodiments, the deuterium-labeled glycolytic substrate comprises deuterated glucose. In preferred embodiments, the deuterated glucose comprises at least 3, 4, 5, 6, or 7 deuterium atoms. Therefore, in some embodiments, the deuterated glucose comprises [²H₇]glucose.

In some embodiments, the DMI step is further used to detect deuterated lactate production in the tissue of the subject by detecting deuterium-induced perturbations of ¹H-NMR chemical shifts. However, in preferred embodiments, the method does not involve detecting deuterated lactate production. This method is less viable, as the shifts are only easily detected using a two-stage experiment with acquisition with and without ²H spin decoupling. This will make the experiment twice as long, and also requires ²H decoupling, which is difficult to implement in humans due to issue with specific absorbance rate (SAR). The power associated with ²H decoupling can trigger SAR safety issues, as it can cause local heating in the subject and is therefore protected against.

Also disclosed herein is a metabolic imaging modality that can be used to screen and follow high-risk patients (i.e., obese patients) and diagnose early on the transition from “benign” NAFLD to NASH and allow prompt intervention to prevent severe liver disease.

Both NAFLD and NASH are characterized by aberrant fatty acid (FA) beta-oxidation in the liver. FA oxidation can be detected using ¹³C based magnetic resonance, but the protocol is cost prohibitive and relies upon a very long examination period for the subject that is clinically intractable. Fatty acid uptake can be estimated with ¹¹C positron emission tomography, but this method cannot directly measure oxidative flux. However, when beta-oxidation proceeds in any living tissue, it generates hydrogen from the precursor FA. If the FA, of any chain length or specific chemistry in regards to double bond positioning, is perdeuterated, then the eliminated hydrogen is instead a deuterium (²H), which causes the generation of HDO instead of H₂O. This HDO signal can be imaged via T₁, weighted ¹H MRI or by ²H based imaging. Deuterium magnetic resonance imaging (DMI) in particular provides the requisite chemical, spatial and temporal resolution needed for detecting HDO signal (De Feyter, H M. et al. et al. Sci Adv 2018 4:eaat7314).

Therefore, detection of HDO following administration of ²H labeled FA precursors, via any route, oral, intravenous, or intraperitoneal, should correlate with increasing or decreasing beta-oxidation. Administration of deuterium-labeled substrates at tracer levels (<200 mg/kg ²H) is well established as safe (Klein, D. Stable Isotopes 1986 378-382; Jones, P J H. & Leatherdale, S T. Clin Sci (Lond). 1991 80(4):277-80; Koletzko, B., et al. Eur J Pediatr. 1997 156 Suppl 1:S12-7). By developing ²H magnetic resonance imaging (MRI) as a sensitive detector of increased hepatic fat oxidation that accompanies the NAFLD/NASH transition, this method has the potential to radically improve patient care and have major clinical translational value for millions of affected individuals.

As disclosed herein, enrichment of ²HOH (i.e. HDO production) after administration of deuterium-labeled FA provides a sensitive marker of liver FA beta-oxidation in vivo, with a possibility for a simplified imaging strategy.

Therefore, disclosed herein is a method for detecting FA beta-oxidation in the liver of a subject, that involves administering to the subject a composition comprising deuterium-labeled FA substrate; and imaging the subject with deuterium magnetic resonance imaging (DMI) to detect hydrogen-deuterium oxide (HDO) in the liver of the subject, wherein detection of elevated HDO in a location within the liver of the subject is an indication of the presence of aberrant FA beta-oxidation in the subject, which is characteristic of NAFLD and NASH. In some embodiments, the DMI comprises ²H-NMR imaging. In some embodiments, the DMI comprises ¹H-NMR imaging that uses T₁ weighting.

As very long chain fatty acids can be oxidized in the peroxisome, which is not thought to share a similar role as mitochondria in NAFLD and NASH, the deuterated fatty acid substrate is preferably a small chain fatty acid (SCFA), a medium-chain fatty acid (MCFA), or a long-chain fatty acid (LCFA), but not a very long chain fatty acid (VLCFA). Therefore, in some embodiments, the fatty acid substrate is a C6-C19 fatty acid, such as a C6, C7, C8, C9, C10, C11, C12, C13, C14, C16, C17, C18, C19, CD20, or CD21 fatty acid.

In some embodiments, the fatty acid substrate is a saturated fatty acid. In some embodiments the fatty acid substrate is an unsaturated fatty acid. In some embodiments, the fatty acid substrate has 0, 1, 2, 3, 4, or 5 double bonds. In some embodiments, the fatty acid substrate is even-chained fatty acids (ECFA). In some embodiments, the fatty acid substrate is odd-chained fatty acids (OCFA). In some embodiments, the fatty acid substrate is a non-esterified fatty acids (NEFA) or free fatty acids (FFA).

In some embodiments, the substrate is composed of a partial or fully deuterated triglyceride (TRG). A TRG contains three fatty acid tails that can be the same length or different lengths. As disclosed herein, the TRG can have any combination of fatty acid tail lengths (e.g. C6-C21), wherein 1, 2, or 3 of the tails are partial or fully deuterated.

In some embodiments, the deuterium-labeled FA substrate comprises deuterated octanoate, which bypasses fatty acid transport mechanisms and is rapidly oxidized.

In some embodiments, the deuterated fatty acid is perdeuterated. In some embodiments, the deuterated fatty acid comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 deuterium atoms (or up to 3×these numbers if a triglyceride).

One advantage of using HDO, e.g. as the surrogate of glucose utilization by cancers, or as a surrogate of beta-oxidation in the liver, is the reduced imaging time that is needed for imaging (DMI). In some embodiments, the imaging step(s) occurs and/or are completed within 60 minutes of the administration of the deuterated tracer, including 5, 6, 7, 8, 9, 10, 11, 12, 13, 14, 15, 16, 17, 18, 19, 20, 21, 22, 23, 24, 25, 26, 27, 39, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes of the administration of the deuterated tracer. As shown herein, the case of glucose, the imaging step(s) preferably occur in a window from 8 to 24 minutes. By choosing the correct imaging window temporally, it is possible to produce images that more accurately estimate metabolic flux without contamination from peripheral metabolism of the administered deuterated tracer. DMI signal to noise ratio (SNR) is defined by the highest peak intensity versus the noise. HDO is the strongest peak in the spectrum. By detecting it, e.g. instead of lactate, the peak with the highest SNR is being used, reducing acquisition time. Also, imaging using the HDO peaks allows other imaging methods than chemical shift imaging (CSI), an imaging sequence that is not easily accelerated by methods like sensitivity encoding for fast MRI (SENSE) (Pruessmann, Klaas P., et al. Magnetic Resonance in Medicine 42.5 1999 952-962.

In some embodiments, chemically selective imaging is accomplished using a suitable MRI pulse sequence. Chemically selective imaging allows the concentration of the starting substrate, e.g. glucose or a FA, to be ascertained apart from the HDO signal intensity or from other downstream metabolic products, e.g. of glucose or fatty acid metabolism.

Chemical shift imaging (CSI) is described in de Feyter, et. Al., Science advances. 2018 4(8):eaat7314, which is incorporated by reference herein for the teaching of this method. Further improvements upon CSI that increase the speed of imaging can be implemented, including spiral-CSI (Adalsteinsson E, et al. Magnetic resonance in medicine. 1998 39(6):889-98, which is incorporated by reference herein for the teaching of this method), CSI using centric sampling (Wilman A H, et al. Magnetic resonance in medicine. 1997 38(5):793-802, which is incorporated by reference herein for the teaching of this method), and CSI using compressed sensing for reconstruction (Lustig M, et al. Magnetic Resonance in Medicine. 2007 58(6):1182-95, which is incorporated by reference herein for the teaching of this method). Echo planar spectroscopic imaging (EPSI) is also possible (Mansfield P. Magnetic Resonance in Medicine. 1984 1(3):370-86, which is incorporated by reference herein for the teaching of this method). Newer implementations of EPSI also exist (Chen A P, et al. Magn Reson Med. 2007 58(6):1099-106; Jakob P M, et al. Magnetic resonance in medicine. 1995 33(4):573-8, which are incorporated by reference herein for the teaching of this method).

An alternative method for producing chemically selective images is to use accumulated phase and multiple sampling windows to produce images which can be combined to produce chemically selective images, commonly referred to as the Dixon method (Dixon W T. Radiology. 1984 153(1):189-94, which is incorporated by reference herein for the teaching of this method). This technique uses two images to produce in phase and out of phase combinations of two metabolites. It can be adapted for 3 resonances as well by using more echoes (Szumowski J, et al. Radiology. 1994 192(2):555-61, which is incorporated by reference herein for the teaching of this method). More sophisticated methods can use even more echoes (Wang J x, et al. Magnetic Resonance in Chemistry. 2016 54(8):665-73, which is incorporated by reference herein for the teaching of this method). This many-echo method is commonly referred to as Iterative decomposition of water and fat with echo asymmetry and least squares estimation (IDEAL) imaging.

An alternative method uses selective excitation of the desired resonance followed by imaging to produce chemically selective images. Long selective pulses can be used to excite the single resonance peak (Meyer C H, et al. Magnetic resonance in medicine. 1990 15(2):287-304, which is incorporated by reference herein for the teaching of this method), but these pulses are subject to RF homogeneity issues. Spectral-spatial pulses are widely used for this application and have superior properties for slice selection (Cunningham C H, et al. Journal of magnetic resonance. 2008 193(1):139-46; Sigfridsson A, et al. Magnetic Resonance in Medicine. 2015 73(5):1713-7, which are incorporated by reference herein for the teaching of this method).

Another method for imaging that could be suitable combines selective excitation with steady-state free precession (SSFP) to rapidly acquire images (Scheffler K, et al. Eur Radiol. 2003 13(11):2409-18, which is incorporated by reference herein for the teaching of this method). These sequences are rapid and produce excellent in plane resolution.

Deuterium MRI is handicapped by the short T₂ of the nucleus. Methods that utilize very short time to echoes could also be applicable in certain circumstances (Hafner S. Magn Reson Imaging. 1994 12(7):1047-51; Robson M D, et al. Journal of computer assisted tomography. 2003 27(6):825-46, which are incorporated by reference herein for the teaching of this method). A similar technique is sweep imaging (ldiyatullin D, et al. Journal of Magnetic Resonance. 2006 181(2):342-9, which is incorporated by reference herein for the teaching of this method).

Each of these methods focus on producing images that separate HDO from any other resonances that would contaminate the image, reducing its diagnostic power.

In some embodiments, the method involves a single imaging step that detects elevated HDO in a tissue, e.g. by comparing it to other tissues. In other embodiments, the method involves imaging the subject at multiple time points in order to determine a rate of HDO enrichment in the tissue of the subject, to detect regional differences in glycolytic flux associated with cancer or other disease. Using kinetic modeling to estimate metabolic turnover has been used to study in vivo metabolism in many contexts (Cerdan S, et al. J Biol Chem. 1990 265(22):12916-26; Patel A B, et al. Proc Natl Acad Sci USA 2005 102(15):5588-93; Sibson N R, et al. Proc Natl Acad Sci USA 1997; 94(6):2699-704, which are incorporated by reference herein for the teaching of this method). These methods measure enrichment as a function of time to measure rates quantitatively. They are endemically more accurate than taking a single image, but require longer scan times for the patient. Patient scan time minimization is essential, as scans longer than 30 minutes are poorly tolerated by the ill.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows lactate isotopomers and HDO (or ²HOH) production from [²H₇]glucose: Schematic representation of lactate isotopomers and HDO production from [²H₇]glucose during the glycolysis and interconversion of F6P and M6P. Deuterium (²H) loss has been shown in the form of ²HOH, NADP²H and NAD²H during glycolysis and the pentose phosphate pathway. Large and small red filled circles represent 2 and 1 deuterium atoms respectively, black filled circles represent hydrogen (¹H) atoms, and black empty circles represent quaternary carbons. Abbreviations are as follows: HK; hexokinase, GK; glucokinase, G6P; glucose-6-phosphate, F6P; fructose-6-phosphate, GPI; glucose-6-phosphate isomerase, PFK; Phosphofructokinase, FBPA; fructose bisphosphate aldolase (or aldolase), G6PDH; glucose-6-phosphate dehydrogenase, PGL; phosphoglucono-lactone, 6PGDH; 6-phosphogluconate dehydrogenase, PMI; phosphomannoseisomerase, R5P; ribulose 5-phosphate, TPI; triose phosphate isomerase, DHAP; dihydroxy acetone phosphate, GA3P; glyceraldehyde 3-phosphate, GAPDH; glyceraldehyde phosphate dehydrogenase, PGK; phosphoglycerate kinase, PGM; phosphoglyceromutase, PEP; phospho-enol pyruvate, PK; pyruvate kinase, and LDH; lactate dehydrogenase. (Note: unlabeled lactate can be produced due to the deuterium loss from the deuterated precursors of lactate in glycolysis).

FIG. 2 shows ²H-decoupling off and on ¹H-NMR spectra of lactate isotopomers: Representative expanded ¹H-NMR of the lactate methyl group in the cell medium of the HUH-7 cancer cells incubated with 5.5 mM [²H₇]glucose. FIG. 2 shows (a)²H-decoupling off, ¹H-NMR spectra, (b)²H-decoupling on, ¹H-NMR spectra, and (c) the difference spectrum of the ²H-decoupling on and off ¹H-NMR spectra. The resolved J-couplings arise from the hydrogen at the C2 position of lactate. The shifts in the methyl resonance position are caused by the isotope effect, with addition of ²H causing a shift to lower frequencies. The broadening of the spectrum in (a) arises from unresolved ¹H-²H J-couplings.

FIG. 3 shows ¹H and ²H NMR spectra of cell media at different incubation time points of the HUH-7 cancer cells: Expanded stacked plot of the {²H}¹H-NMR and {¹H}²H-NMR spectra of the HUH-7 cell line incubated with 5.5 mM [²H₇]glucose. Cell media withdrawn at (a) 0 min, (b) 20 min, (c) 1h, (d) 2h and (e) 5h incubation time points from cultures of HUH-7 cancer cells. Labeling in the (e) spectrum of panel A showing lactate-CH and lactate-CH₃, lactate-CH₂D and lactate-CHD₂ isotopomer shifts due to the ²H incorporation at the methyl position. In panel B, labeling on each peak in the (e) spectrum showing the resonances arise from the pyrazine-D₄, HDO, residual [²H₇]glucose, and ²H-lactate. (Note: the intensity scale of glucose and lactate region is 10×increased).

FIG. 4 shows Linearity graph of the lactate isotopomers effluxed from HUH-7 cells into the cell media: Lactate efflux from HUH-7 cancer cells into the cell culture medium. Green, red, and blue curves demonstrating the relative rate of the production of the Lac—CH₃, Lac—CH₂D and Lac—CHD₂ with respect to the incubation time. Linear fits (dotted lines) were suitable models of lactate efflux from the cells. Even after switching media, unlabeled lactate continues to be produced. The concentrations are plotted and are derived from data normalized for differential T₁s and the number of hydrogens leading to the resonances.

FIG. 5 shows relative pool size contribution of lactate isotopomers: Percent contribution of the lactate isotopomers to the lactate efflux pool size from the HUH-7 cells into the cell culture medium. Contribution of the Lac—CH3 decreases whereas Lac—CH2D and Lac—CHD2 increases. Fully deuterated lactate cannot be produced using [²H₇]glucose.

FIGS. 6A and 6B show [²H₇]glucose consumption and HDO production by HUH-7 and AML12 cells: Plot of (a) [²H₇]glucose consumption and (b) HDO evolution by AML12 cells (n=3, red circles) and HUH-7 cells (n=4, black squares) during incubation in the DMEM with 5.5 mM [²H₇]glucose. These results were calculated from ²H-NMR data of each of the cell media samples including blank cell media sample withdrawn at 0 min time point. Both graphs clearly indicate that the evolution of HDO and consumption of [²H₇]glucose is very high in the case of HUH-7 cancer cell lines.

FIG. 7 is a ²H-NMR spectra of the AML12 cell media at different time points: Stacked plot of the ¹H-decoupled ²H-NMR spectra of the AML12 cell line incubated with 5.5 mM [²H₇]glucose. Cell media withdrawn at (a) 0 min, (b) 20 min, (c) 1 h, (d) 2 h and (e) 5 h time points from cultures of AML12 cancer cells. Labeling on each peak in the (e) spectrum showing the resonances arise from the pyrazine-D4, HDO, residual [²H₇]glucose, and ²H-lactate.

FIG. 8 is an inversion recovery spectra of pyrazine and lactate isotopomers: Stacked plot of the T₁-inversion recovery ¹H-NMR spectra recorded with ²H-decoupling during acquisition. Inversion recovery delays (T) used to acquire the FIDs have been indicated in the front of respective spectrum. A and B marks represent the pyrazine and lactate isotopomers peak respectively.

FIG. 9 shows proton T₁ relaxation times of the lactate isotopomers: Longitudinal relaxation time (T₁) fitting curves and calculated T₁ relaxation times for (a) lac-CH3, (b) lac-CH2D and (c) lac-CHD2 isotopomers. T₁ relaxation times were calculated using the normalized inversion recovery data and the standard inversion recovery equation.

FIG. 10 is a deuterium T₁-inversion recovery spectra: Stacked plot of the T₁-inversion recovery ²H-NMR spectra, showing the pyrazine-D₄ and HDO resonances. Inversion recovery delays (T) used to acquire the FIDs have been indicated in the front of respective spectrum. T₁ relaxation times were calculated for HDO and pyrazine-D₄ and found to be 0.49s and 0.45s respectively.

FIG. 11 shows a representative time series ²H NMR spectra obtained from rat brain. ²H NMR spectra represent (a) natural abundance deuterium signal before [²H₇]glucose infusion, (b) 2 min post-infusion, (c) 24 min post-infusion, (d) 35 min post-infusion, (e) 120 min post-infusion and (f) 150 min post-[2H₇]glucose infusion. Each in vivo ²H NMR spectrum was obtained from a single 256 scan acquisition. ²H resonance assignments are for HDO, [²H₇]glucose including the anomeric deuterium, deuterated-glx (glutamate/glutamine), and deuterated-lactate.

FIGS. 12A to 12C are time series for the signals of [²H₇]glucose, HDO, glx (glutamate/glutamine), and lactate was calculated using the fitted areas for the ²H MRS data from rat brain. FIG. 12A shows a series of data points indicating the absorption and utilization of [²H₇]glucose for production of HDO, deuterated glx and deuterated lactate. [²H₇]glucose (blue diamonds) increases in signal intensity on delivery and subsequently decreases in its signal intensity with time. HDO (red squares) shows a continuous increase in the signal intensity post-[²H₇]glucose infusion. Glx (black circles) and deuterated lactate (green triangles) show an increase in the signal intensity and slight decrease at the end of the experiment. FIG. 12B shows the 5 time points (5 min, 15 min, 38 min, 2 hrs, and 2.5 hrs) of the dynamic change in the signals of [²H₇]glucose, HDO, deuterated glx, and deuterated lactate. HDO (red diamonds) concentration continuously increased with time, even after the cerebral glucose was apparently consumed. Body water enrichment of HDO was approximately 1.8×greater than the brain concentration at the final time point, indicating that the continual increase was likely due to continued glx and lactate turnover, as well as extra-cerebral metabolism. The plots in FIG. 12C for HDO+lactate to glx (red line) and HDO to glx (blue line) ratio with time show a stable ratio from ˜8 to 24 minutes. (Note:Natural abundance deuterated HDO signal intensity was subtracted from the HDO signal of each spectrum. Data is represented as mean±standard error of mean (SEM)). This temporal region is where HDO generation exactly mimics lactate and glutamate/glutamine production, producing quantitative estimates of metabolic flux using HDO without contamination from peripheral metabolism.

FIGS. 13A to 13H show shows DMRI images showing the consumption of [²H₇]glucose and production of HDO in the brain. FIG. 13A is a proton intensity image of brain and a container for tap water as a reference for deuterium surface coil positioning.

FIGS. 13B and 13C show the natural abundance deuterium pre-[²H₇]glucose infusion (FIG. 13B), and an overlaid image (FIG. 13C) of the proton (grey) and deuterium (green) images.

FIG. 13E represents the deuterium intensity image after 50 min of [²H₇]glucose infusion and

FIG. 13F is the overlaid images of proton (grey) and deuterium (green) intensity images.

FIG. 13H represents the deuterium image after 2.5 hrs of the [²H₇]glucose infusion and FIG. 13I is the overlaid images of proton (grey) and deuterium (green) intensity images. Note:

FIGS. 13A, 13E, and 13G are proton intensity images obtained before every corresponding deuterium image for co-registration of proton and deuterium images. Size of deuterium images were scaled by interpolation to the proton image for co-registration of the images.

FIGS. 14A to 14G show multi-gradient echo (MGE) imaging (Dixon imaging) to separate the contribution of [²H₇]glucose and HDO to DMRI image of the rat brain. FIGS. 14A to 14C show the proton intensity image of brain (FIG. 14A), the natural abundance HDO pre-[²H₇]glucose infusion image (FIG. 14B), and the overlaid image of the proton (grey) and deuterium (green) images (FIG. 14C). FIG. 14D represents the deuterium image of HDO-only and FIG. 14E is the overlaid images of proton (grey) and deuterium (green) images due to HDO-only in the rat brain. FIG. 14F represents the deuterium image from the contribution of [²H₇]glucose-only and FIG. 14G is the overlaid image of proton (grey) and deuterium (green) [²H₇]glucose-only images.

FIG. 15 is a schematic representation of the production of deuterated lactate, glutamate/glutamine (glx), and HDO from [²H₇]glucose during glycolysis and the TCA cycle. Deuterium (²H) loss has been shown in the form of ²HOH, and NAD²H. Small and large red filled circles represent 1 and 2 deuterium atoms respectively, black filled and empty circles represent hydrogen atoms and quaternary carbons, respectively. Abbreviations of the glycolytic intermediates are as follows: G6P; glucose-6-phosphate, F6P; fructose-6-phosphate, M6P; mannose-6-phosphate, DHAP; dihydroxy acetone phosphate, GA3P; glyceraldehyde 3-phosphate, PEP; phospho-enol pyruvate. Enzymes involved in glycolysis and TCA cycle are as follows: (1) hexokinase (HK), (2) glucokinase (GK), (3) glucose-6-phosphate isomerase (GPI), (4) phosphomannoseisomerase (PMI), (5) Phosphofructokinase (PFK), (6) fructose bisphosphate aldolase (FBPA or aldolase), (7) triose phosphate isomerase (TPI), (8) glyceraldehyde phosphate dehydrogenase (GAPDH), (9) phosphoglycerate kinase (PGK), (10) Phosphoglyceromutase (PGM), (11) Enolase (12) pyruvate kinase (PK), (13) lactate dehydrogenase (LDH), (14) alanine aminotransferase (ALT) (15) pyruvate dehydrogenase (PDH), (16) glutamate dehydrogenase (GDH), and (17) glutamine synthetase (GS). (Note: unlabeled lactate, pyruvate, glutamate, and glutamine can be produced due to deuterium loss from the deuterated precursors in glycolysis and by multiple turns of TCA cycle).

FIG. 16 is a correlation plot between [²H₇]glucose consumption and HDO production. The delivery of [²H₇]glucose into the rat brain peaks at approximately 850 units of glucose consumption, corresponding to approximately 10 minutes post infusion. The rate of [²H₇]glucose consumption is matched by HDO production from 10 to 24 minutes (first dotted line). The second dotted line shows that HDO appearance in the brain remains linear after this first window, but the slope changes as glucose consumption no longer precisely matches the HDO production.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

Disclosed herein is a method for detecting a tumor in a subject, that involves administering to the subject a composition comprising a deuterium-labeled glycolytic substrate; and imaging the subject with deuterium magnetic resonance imaging (DMI) to detect hydrogen-deuterium oxide (HDO) in tissues of the subject, wherein detection of elevated HDO in a location within the tissue of the subject is an indication of the presence of a tumor in the tissue of the subject. The likelihood that D₂O can be formed by metabolism or exchange is very low, but its formation would not preclude use of this technology as stated.

Also disclosed herein is a method for detecting fatty acid beta-oxidation in the liver of a subject, that involves administering to the subject a composition comprising a deuterium-labeled fatty acid substrate; and imaging the subject with deuterium magnetic resonance imaging (DMI) to detect hydrogen-deuterium oxide (HDO) in liver of the subject, wherein detection of elevated HDO in a location within the tissue of the subject is an indication of the presence of fatty acid beta-oxidation of the subject.

In some embodiments, the deuterium-labeled substrate is administered to the subject as an oral composition. Compositions for oral administration include powders or granules, suspensions or solutions in water or non-aqueous media, capsules, sachets, or tablets. Thickeners, flavorings, diluents, emulsifiers, dispersing aids or binders may be desirable.

Formulations can be prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, matrix-forming compositions and coating compositions.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

In some embodiments, deuterium-labeled substrate is administered in a dose equivalent to parenteral administration of about 1 mg to about 10 g per kg of body weight, including about 1 mg to about 1 g, about 1 g to about 10 g, about 10 mg to about 1 g, and about 100 mg to about 10 g.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained. See, e.g., U.S. Pat. No. 3,610,795, which is incorporated by reference herein.

Deuterium metabolic imaging (DMI) is a noninvasive approach that combines deuterium magnetic resonance spectroscopic imaging with oral intake or intravenous infusion of nonradioactive ²H-labeled substrates to generate three-dimensional metabolic maps. Deuterium NMR (²H-NMR) is NMR spectroscopy of deuterium (²H or D), an isotope of hydrogen. Deuterium is an isotope with spin=1, unlike hydrogen which is spin=½.

Deuterium NMR has a range of chemical shift similar to proton NMR but with poorer resolution.

Deuterium usually yields broad signals whose line-width typically varies between a few hertz and a few kilohertz. The spectrum has the same narrow chemical shift range as for H but with a lower resolution and lower sensitivity. Deuterium-deuterium couplings are about 40 times smaller than proton-proton couplings and are therefore not observed.

A number of embodiments of the invention have been described.

Nevertheless, it will be understood that various modifications may be made without departing from the spirit and scope of the invention. Accordingly, other embodiments are within the scope of the following claims.

### EXAMPLES

### Example 1: HDO Production from [2H₇]Glucose Quantitatively Identifies Warburg Metabolism

Introduction

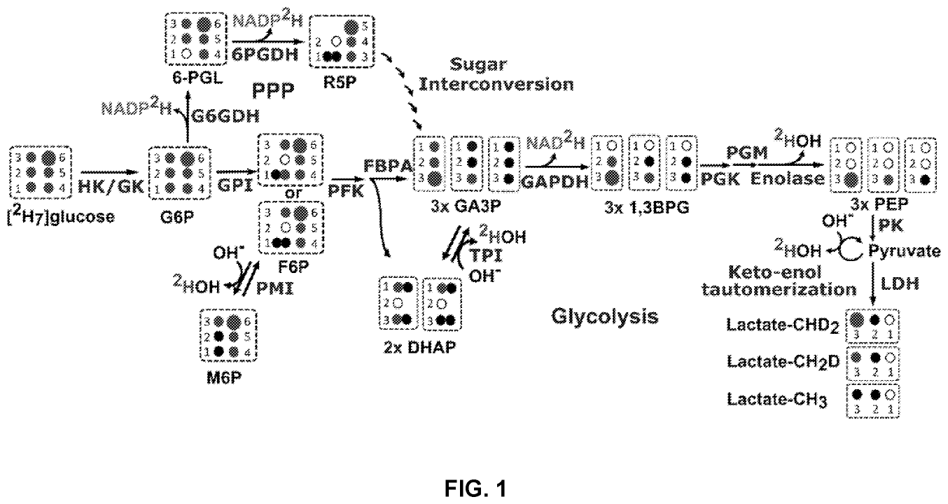

In this Example, highly sensitive ¹H-NMR was used to measure the production of lactate isotopomers and ²H-NMR to measure HDO production and residual [²H₇]glucose (eventually cellular glucose uptake) in the cell culture medium. The fate of the ²H tracer for lactate isotopomers, HDO production, and loss of ²H atoms from [²H₇]glucose during glycolysis, sugar isomerization and pentose phosphate pathway (PPP) can be easily understood from FIG. 1. Although deuterium J-coupled satellites are not well distinguished in ¹H-NMR spectra due to the very low coupling constant of 2J_{H-D}, ²H-decoupling and difference spectra can overcome these limitations. Utilizing the small isotope shifts of mono-deuterated and di-deuterated lactate in the ¹H-NMR spectrum, lactate-CH₂D and lactate-CHD₂ resonances are resolved, enabling a novel methodology for producing metabolic flux measurements by MR. Simultaneously, HDO production rate measurement enhances the sensitivity of the ²H-NMR based metabolic flux measurements, as it records a response linear with lactate production, producing a metric of Warburg metabolism with higher sensitivity than the estimate made with lactate alone Additionally, a strong correlation between cellular glucose uptake and HDO production could be a diagnostic modality for in vivo imaging.

Methods

Cell Line, Chemicals, and Media

HUH-7 hepatocellular carcinoma cells were received. AML12 cell lines were purchased from the American Type Culture Collection (Manassas, Va.). Pyrazine, Pyrazine-D₄ and [1,2,3,4,5,6,6-²H₇]-D-glucose ([²H₇]glucose) were purchased from Sigma Aldrich, (St. Louis, Mo., USA).

Cell Culture

HUH-7 or AML12 cell lines were cultured in a complete growth medium made up of Dulbecco's Modified Eagle Medium (DMEM) with 10% v/v FBS, 10,000 units/mL penicillin, 10,000 μg/mL streptomycin, 25 μg/mL amphotericin B and 5.5 mM [²H₇]glucose. Cell lines were maintained at 37° C. in a 5% CO₂ in air atmosphere in an air-jacketed incubator (Heracell Vios 160i, Thermo Scientific, Waltham, Mass.). Growth media was replaced every 3 days, and subcultured 1:5 at 80% confluence. 25 cm² cell culture plates, containing 15 million HUH-7 or AML12 cells at 70-80% confluence, were washed once with warm PBS and incubated with 4 mL each of DMEM with deuterated tracer for 5h, withdrawing 100 μL aliquots at 20 min, 1h, 2h, and 5h.

Sample Preparation

No extraction was performed on the cell media samples. Pyrazine and pyrazine-D₄ were mixed to achieve 5 mM concentration of each of the standards in each cell media samples of HUH-7 and AML12 cells. Pyrazine and pyrazine-D₄ served as an internal standard to quantify the lactate isotopomers from ¹H-NMR data and HDO as well as residual [²H₇]glucose from ²H-NMR data respectively. 40 μL of cell medium was transferred into 1.7 mm NMR tubes for NMR analysis. ¹H-NMR Spectroscopy

All NMR spectra were recorded on a 600.23 MHz NMR, equipped with a 1.7 mm TCI CryoProbe and Avance Neo Console (Bruker Biospin). All NMR experiments were set up in the lock off followed by sweep off mode. ²H-decoupling off and on ¹H-NMR spectra were acquired with a sequence that alternated decoupling with each scan. Relaxation delay (d1) of 1.5s and acquisition time (AQ) of 2s (3.5s of recycling time, Tr) and a 90° pulse of 10.50 μs was used for acquisition of each of the spectra. The WET method containing selective pulses was applied on the strong water resonance (M o, H. & Raftery, D. J. Biomol. NMR 2008 41:105-111). Waltz16 ²H-decoupling sequence consists of 90° pulse of 250 μs with decoupling power of 1.72watts, producing B, of 1 kHz during the acquisition period. 13,157 complex data points were acquired with the spectral width of 11 ppm. 128 scans were used to acquire each spectrum. 4 dummy scans were also used for equilibration of the spin states prior to acquisition. A modified T₁ inversion recovery pulse sequence with ²H-decoupling on during the acquisition was used to measure the T₁ of the lactate isotopomers and pyrazine standard. Relaxation delay (d1) of 50s and inversion recovery delays (T) of 0.001, 0.6, 0.9, 1.5, 2.0, 3.0, 6.0, 9.0, 14.0, 20.0, 30.0 and 40.0s were used to determine the T₁ of lactate isotopomers.

²H-NMR spectroscopy

The deuterium lock channel was used to acquire the ¹H-decoupled ²H-NMR spectrum at 92.13 MHz resonant frequency. Relaxation delay (d1) of 2s and acquisition time (AQ) of 1s (3s of recycling time, Tr) with a 90° pulse was used to acquire each of the ²H-NMR spectra. 1086 complex data points were digitized with the spectral width of 11 ppm using 1024 scans for each of the 5 FIDs (5120 scans) for each of the samples. T₁ inversion recovery pulse sequence was used to calculate the T₁ relaxation times of HDO and pyrazine-D₄ standard. Relaxation delay (d1) of 5s, acquisition time (AQ) of 1s and inversion recovery delays (T) of 0.001, 0.1, 0.4, 0.8, 1.6, and 3.2s were used to acquire an array of FIDs to calculate the T₁ of HDO and pyrazine-D₄. All NMR experiments were carried out at the room temperature (25° C.). ¹H-NMR Data Processing and Quantification of Lactate Isotopomers

NMR data analysis was performed using MestReNova v14.0.1-23284 (Mestrelab Research S. L.). For processing of the ¹H-NMR spectrum, the spectra were zero-filled to 32,768 points with an exponential window function of 0.3 Hz before the Fourier Transform (FT). Manual phase and automatic spline baseline correction were performed on each of the spectra. ²H-decoupled ¹H-NMR spectra of each time point were used for the extraction of the areas of lactate isotopomers. The MestReNova line fitting tool was used to extract the peak areas of the slightly overlapped signals for quantification of the lactate isotopomers. A T₁ correction factor for each lactate isotopomer was used to calculate concentrations using the ¹H-NMR data (Bharti, S. K. et al. Metabolomics 4:367-376 (2008)).

²H-NMR Data Processing and Quantification of HDO and Residual [²H₇]Glucose

²H-NMR spectra were processed with zero-filling of FID to 4096 points followed by exponential window function of 1 Hz before the Fourier Transform (FT). Each spectrum was manually phase corrected and followed by automatic spline baseline correction. Five FIDs were acquired for each of the samples and all five ²H-NMR spectra were aligned and summed-up to compensate the peak shifting due to magnetic field drifting over the course of time. Concentrations of HDO and residual [²H₇]glucose in the cell media were calculated using peak areas of the internal standard pyrazine-D₄ (concentration 5 mM), HDO and residual [²H₇]glucose, normalized with the number of deuterons responsible for corresponded resonances. Baseline ²H content of ultrapure water was also measured with laser cavity ring-down spectroscopy (CRDS) (Picarro Science Instruments).

Results ¹H-NMR spectra of the cell culture media

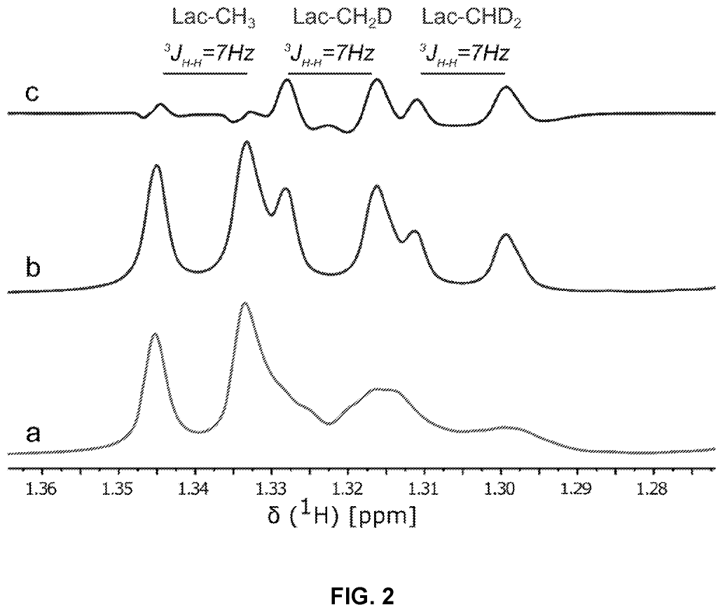

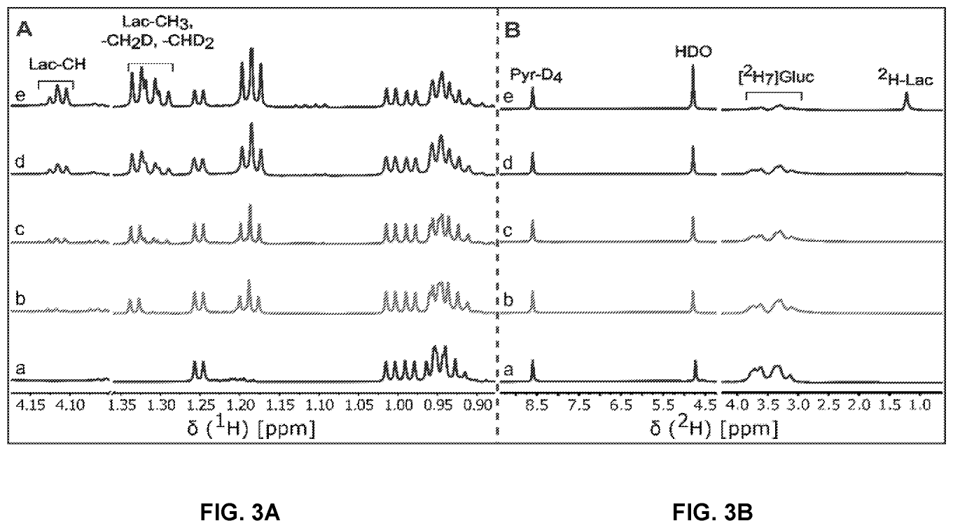

1H-NMR spectra in ²H-decoupling off and on mode were recorded for all of the withdrawn cell media. NMR spectra from the 5 hour time point show pronounced isotope shifts for lactate-CH₂D and lactate-CHD₂ isotopomers (FIGS. 2A & 2B). Methyl signals of the deuterium-enriched lactate appear broad in ²H-decoupling-off ¹H-NMR spectrum, whereas methyl signals appear as sharp doublets in ¹H-NMR spectrum acquired with ²H-decoupling on during the acquisition. Each replacement of a methyl proton with a deuteron introduces an isotope shift in the ¹H spectrum of ˜10 Hz. The difference spectrum of lactate-CH₂D and lactate-CHD₂ show the (³J_{H—H}) of ˜7.00 Hz clearly. The lactate-CH₃ doublet is a very small antiphase doublet due to small differences in peak position between blocks prior to subtraction. The metabolic scheme shown in the FIG. 1 indicates the formation of mono-deuterated and di-deuterated lactate isotopomers from [²H₇]glucose, clearly observed in the {²H}¹H spectrum (FIG. 2B). FIG. 3A shows the stacked plot of the {²H}¹H-NMR spectra of cell media samples of the different time points. Intensity of ¹H-NMR signals for all of the lactate isotopomers increases with incubation time.

²H-NMR Spectra of Cell Culture Media

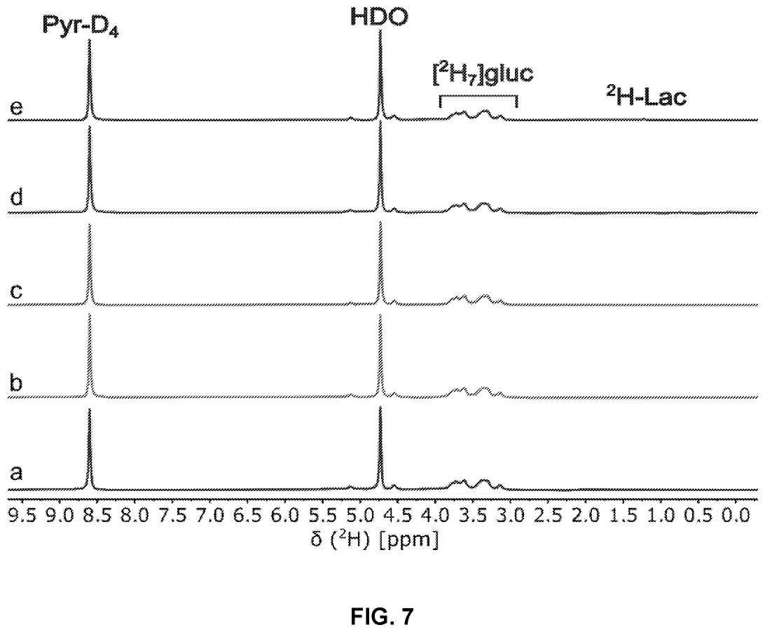

Proton-decoupled ²H-NMR spectra were recorded for all of the withdrawn cell media samples for HUH-7 and AML12 cell lines. HDO signal intensity increases with incubation time period whereas [²H₇]glucose decreases rapidly for HUH-7 cells as indicated by stacked plot of ²H-NMR spectra in FIG. 3B. The stacked plot of ²H-NMR spectra of AML12 cell media samples at different time points indicated slow increase of HDO and slow decrease of [²H₇]glucose (FIG. 7). Qualitatively, both stacked plots demonstrated that the cellular [²H₇]glucose uptake and HDO production rate is very high in HUH-7 cancer cells compared to AML12 cells, reflecting the presence of Warburg metabolism.

Longitudinal Relaxation Time (T₁) Measurement of the Compounds

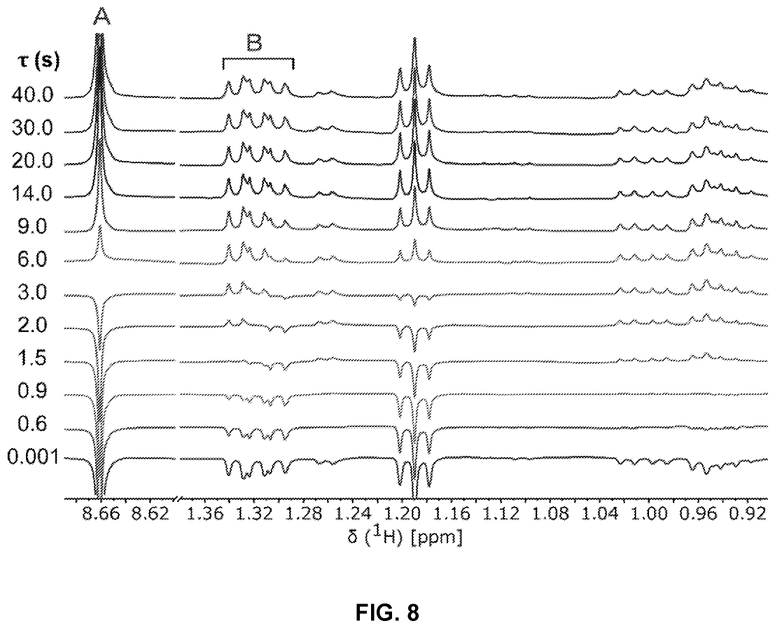

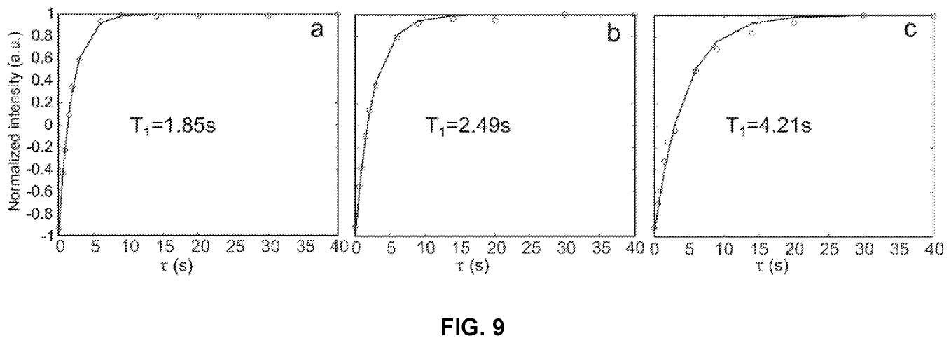

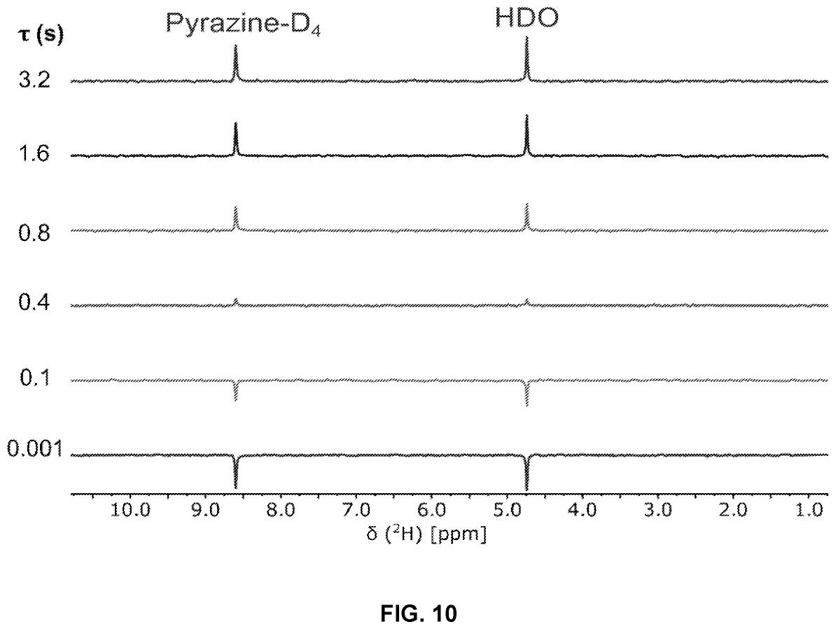

Once the lactate isotopomers were identified, it would be expected to observe differential changes in T₁ due to a loss of ¹H dipolar relaxation. T₁ inversion recovery experiments were performed in the ²H-decoupling on mode to calculate the T₁ relaxation time of the pyrazine and lactate isotopomers. FIG. 8 shows the stacked plot of the inversion recovery data. A and B marks on the uppermost spectrum represent the pyrazine standard and methyl signals of the lactate isotopomers respectively. Integral areas of each of the lactate isotopomers and pyrazine standard were extracted to calculate the T₁ relaxation times. Data was fit to the standard T₁ equation, M_{z}(t)=M₀(1-2*^{T/T}₁), to extract the T₁s. FIG. 9 shows fitting curves for T₁ measurement and T₁ values of lactate—CH₃, lactate—CH₂D and lactate—CHD₂, are 1.85, 2.49 and 4.21s respectively at this field strength. T₁ of the pyrazine standard was found to be 6.45s. T₁ relaxation time of the HDO and pyrazine-D₄ standard were calculated using the inversion recovery ²H-NMR data (FIG. 10) and found to be 0.49s and 0.45s respectively.

Quantification of the Lactate Isotopomers

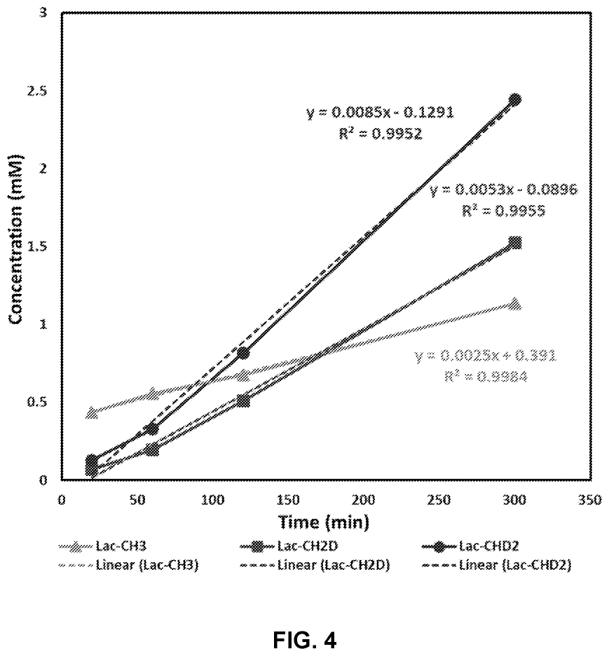

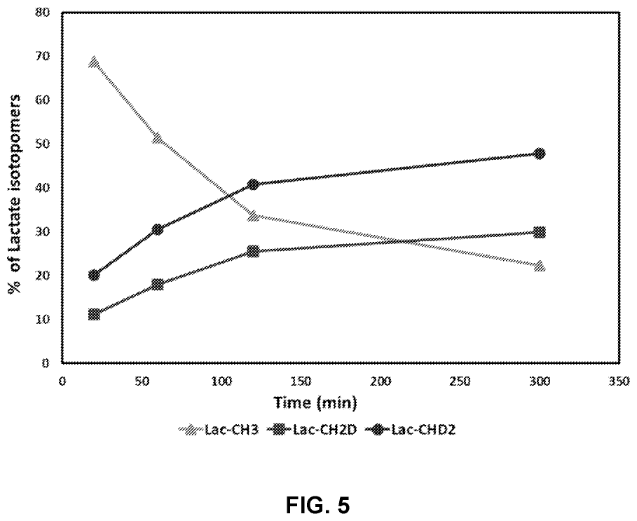

Pyrazine was used as an internal standard to quantify each of the lactate isotopomers. Peak areas of the isotopomers were extracted from each of the {²H}¹H—NMR spectra of the HUH-7 cells at different incubation times. After correction for T₁ and for the number of Hs leading to each resonance, the concentration of the isotopomers was plotted (FIG. 4). All lactate isotopomers in the media increase in concentration with incubation time period. Linearity for lactate isotopomer production and efflux from HUH-7 cells to culture medium is excellent with R² of 0.99. Percent contributions of all lactate isotopomers to the pool size of lactate have been shown in the FIG. 5. As expected, the contribution of labeled lactate increases with time whereas the natural abundance lactate contribution to pool size decreases as incubation time period increases. At the 5 hour time point, the measured lactate—CHD₂ isotopomer was 1.6 fold higher in concentration than the —CH₂D isotopomer.

Quantification of the HDO and Residual [²H₇]Glucose

Pyrazine-D₄ was used as an internal standard to quantify the HDO production and residual [²H₇]glucose in each of the cell media samples of HUH-7 and AML12 cell lines. Peak areas of pyrazine-D₄, HDO and residual [²H₇]glucose was used to measure the concentrations of HDO and residual [²H₇]glucose with respect to the 5 mM pyrazine-D₄ internal standard. Cellular [²H₇]glucose consumption is calculated from the residual [²H₇]glucose in the cell media whereas HDO production was corrected for the natural abundance HDO in the cell media samples of each time points. Cavity ring-down spectroscopy (CRDS) measurements of starting water ²H content provided a secondary internal standard.

### DISCUSSION

The ¹H—NMR spectrum has a complicated pattern due to the very small geminal couplings (²J_{H-D}) and vicinal couplings (³J_{H—H}) of methyls of the partially deuterated lactate isotopomers (FIG. 2A). Since J_{H-D} is roughly ⅙^{th} of J_{H—H}(γD/γH=1/6.48), most of the couplings appeared as a line broadening in ²H-decoupling-off ¹H—NMR spectrum. To resolve methyl signals, ²H-decoupling was implemented during the acquisition period to remove the small geminal couplings (²J_{H-D}). The {²H}¹H—NMR spectrum in FIG. 2B shows a distinguished pattern of the remaining vicinal couplings (³J_{H—H}) of ˜7.00 Hz for each of the methyl signals of deuterated lactate as well as unlabeled lactate. Deuterium-induced perturbations of ¹H—NMR chemical shifts (isotope shift) allows resolution of the lactate isotopomers. The extent of isotope shift of methyl proton increases in the order CH₃<CH₂D<CHD₂, which produces a measurable peak separation (Schah-Mohammedi, P. et al. J. Am. Chem. Soc. 122:12878-12879 (2000)). Utilizing the advantage of the isotope shifts we could resolve the lactate isotopomers in {²H}¹H—NMR spectra (FIG. 2B). Metabolism of the [²H₇]glucose produces lactate—CH₂D and lactate—CHD₂, which is also evident from the ¹H-NMR spectrum in the FIG. 2b. To measure the kinetics of lactate production from HUH-7 cells, cell media was withdrawn at 5 time points including a blank at 0 min. No trace of lactate was found in blank cell media, which is evident from the spectrum shown in FIG. 3a. After 20 min of incubation of HUH-7 cells, efflux of unlabeled lactate signals can be observed and it was presumably derived from unlabeled glucose or pyruvate that remained in the HUH-7 cells (FIG. 3B). However, unlabeled lactate is continuously produced due to the loss of ²H from the lactate precursors during glycolysis (FIG. 1). As incubation time proceeds, appearance of ²H-lactate dominated the total lactate methyl region, whereas the lactate—CH signal pattern becomes more complicated due to multiple vicinal couplings (³J_{H—H}) of lactate-CH with lactate—CH₃, —CH₂D, and —CHD₂. It has been demonstrated in the metabolic scheme of the conversion of exogenous [²H₇]glucose to lactate via glycolysis, that the lactate—CH is not deuterated (FIG. 1). Hence, methyl signals for all of the lactate isotopomers are expected to be doublets in {²H}¹H—NMR spectra, which can be clearly seen in panel B (FIG. 3B-D).

Proton relaxation times of the lactate isotopomers increases incrementally with the number of deuterons (FIG. 9). The dipole-dipole interaction is the dominant mechanism in the proton T₁ relaxation time, and introducing lower-γ nuclei lengthens the relaxation of nearby ¹H nuclei. Therefore, the intramolecular dipole-dipole interactions between the spin % nuclei, i.e. proton-proton, dominate any J-quadrupolar cross-relaxation term. With this effect T₁ relaxation times of the lactate isotopomers increase in the order of lactate—CH₃, —CH₂D and —CHD₂. Concentration of each of the deuterated lactate isotopomers increases with incubation time. At the start of the cell culture incubation, lactate—CH₃ derived from endogenous source was effluxed into the cell media. It is expected that HUH-7 cells have taken some time to transport and metabolize exogenous [²H₇]glucose. Production of labeled lactate rapidly increases after 1h of incubation time. The rate of production of lactate-CH₃, lactate—CH₂D and lactate—CHD₂ was calculated using linear regression and found to be 0.17, 0.35, and 0.57 micromol/10⁶ cells/min respectively. One of the key enzymes in glycolysis is aldolase (FIG. 1) which produces glyceraldehyde 3-phosphate (GA3P) and dihydroxyacetone phosphate (DHAP) (Tittmann, K. Bioorganic Chemistry 57:263-280 (2014); Harris, T. K., et al. Biochemistry 37(47):16828-38 (1998)). GA3P proceeds down glycolysis and ends up labeling lactate—CHD₂. DHAP converts into GA3P by activity of triose phosphate isomerase (TPI) and then produces lactate—CH₂D through the rest of glycolysis. Theoretically, the relative concentration and production rate of the lactate—CHD₂ and lactate-CH₂D should be equal, but we found a differential concentration of the lactate isotopomers from [²H₇]glucose.

Ben-Yoseph et. al. found a significant loss of ²H atoms from C1 and C6 of the [1-²H]glucose and [6,6-²H₂]glucose respectively. Loss of ²H from C6 of glucose was mainly due to catalytic action of pyruvate kinase (PK) whereas loss of ²H from C1 of glucose was due to both PK and phosphomannose isomerase (PMI) (FIG. 1) (Ben-Yoseph, O., et al. Magnetic Resonance in Medicine 32:405-409 (1994)). De Feyter et. al. also showed that the loss of ²H atoms for ²H-lactate was ˜8%, while using [6,6-²H₂]glucose as a tracer (Feyter, H. M. De et al. Deuterium metabolic imaging (DMI) for MRI-based 3D mapping of metabolism in vivo. Sci Adv. 4(8):1-12 (2018)). The rate of ²H loss from lactate—CH₂D and lactate—CHD₂ precursors could be different due to deuterium secondary isotope effects; typical isotope effects may result in 10-15% higher loss of ²H from lactate—CH₂D precursor (Ross, B. D., et al. Biochemical Journal 302:31-38 (1994); Saur, W. K., et al. Biochemistry 7:3537-3546 (1968)). The presence of a ²H kinetic isotope effect at TPI, results in more ²H liberation from the DHAP.

Pentose phosphate pathway (PPP) is another possible source of diversion of ²H from C1 of [²H₇]glucose derived intermediates (FIG. 1) (Ross, B. D., et al. Biochemical Journal 302:31-38 (1994); Cosgrove, M. S., et al. Biochemistry 37:2759-2767 (1998)). Due to these various factors involved in the metabolism of [²H₇]glucose, a small amount of lactate—CH₃ was continuously effluxed into the cell media from HUH-7 cells (FIG. 4).

Relative percent contributions of lactate isotopomers to the efflux pool size are shown in FIG. 5. At 20 min time point, contribution of lactate—CH₃was highest and labeled lactate contribution was very low. Labeled lactate contribution rapidly increased whereas lactate—CH₃ contribution decreased with incubation time. Labeled lactate contribution to the pool size was highest at 5h and at this point lactate—CH₃ has the lowest contribution to the total pool size. It should be kept in mind that the lactate—CHD₂ contribution to the pool size is always higher than that of the lactate—CH₂D.

The mechanism of each step of glycolysis, indicated that HDO would be produced by triose phosphate isomerase (TPI) from the C2 position of GA3P and DHAP, within the course of direct metabolism of one equivalent of [²H₇]glucose to pyruvate. HDO production from the C1/C2 positions can occur due to the interconversion of F6P and M6P via phosphomannoseisomerase (PMI) (Chandramouli, V., et al. American Journal of Physiology-Endocrinology and Metabolism 277:E717-E723 (1999)). Enolase activity on phospho-enol pyruvate (PEP) precursor and keto-enol tautomerization in pyruvate are also prominent sites of HDO production during glycolysis (FIG. 1) (De Feyter, H. M. et al. Deuterium metabolic imaging (DMI) for MRI-based 3D mapping of metabolism in vivo. Sci Adv 4:eaat7314 (2018); Harris, T. K., et al. Biochemistry 37(47)16828-38 (1998); Cosgrove, M. S., et al. Biochemistry 37:2759-2767 (1998); Read, J. et al. Journal of Molecular Biology 309:447-463 (2001); Tittmann, K. et al. Bioorganic Chemistry 57:263-280 (2014); Lorentzen, E., et al. Biochemistry 44:4222-4229 (2005); Rieder, S. V & Rose, I. A. The Journal of biological chemistry 234:1007-10 (1959); Reis, M. et al. Physical Chemistry Chemical Physics 15:3772-3785 (2013); Duquerroy, S., et al. Biochemistry 34:12513-12523 (1995); Lebioda, L. & Stec, B. Biochemistry 30:2817-2822 (1991); Hanau, S., et al. Journal of Biological Chemistry 285:21366-21371 (2010); Ben-Yoseph, O., et al. Magn. Reson. Med. 32:405-409 (1994). Apart from glycolysis, deuterium can also be lost to solvent water in the form of HDO in several reactions in the TCA cycle (Aguayo, J. B., et al. Experimental Eye Research 47:337-343 (1988); Lu, M., et al. J Cereb Blood Flow Metab 37:3518-3530 (2017)).

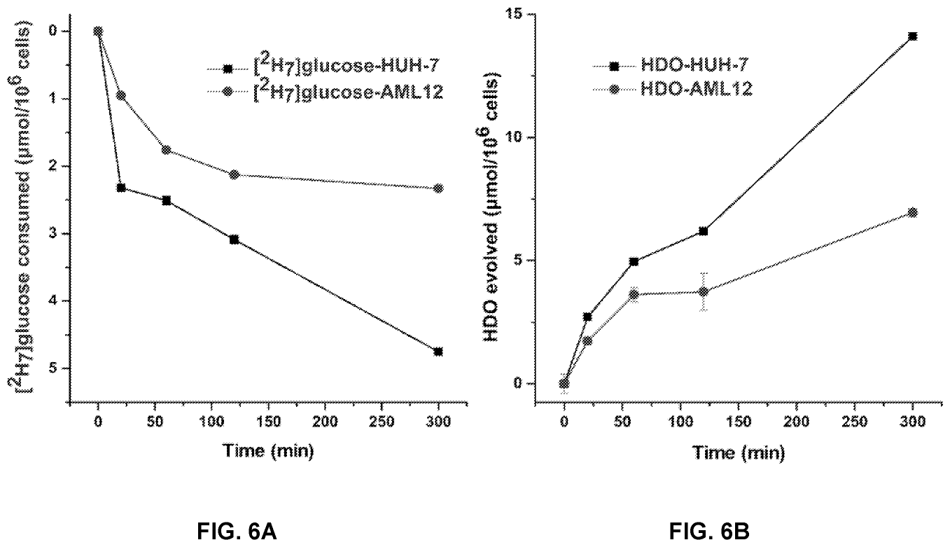

The results indicate that metabolism of [²H₇]glucose produces an increase in the HDO signal that correlates with cellular glucose uptake in the HUH7 cells, and a dramatic difference in HDO production has been observed between HUH-7 cancer cells and background precursor cells (AML12 cells) (FIG. 6). To prove the correlation of cellular [²H₇]glucose uptake versus ²H-lactate and HDO production, we calculated the ²H mass balance at 5 hour time point. Approximately 96% ²H atoms of [²H₇]glucose were incorporated into ²H-lactate and HDO, strongly suggesting that the ²H-based cancer metabolism study is highly efficient and very convenient (Table 1). [²H₇]glucose uptake and HDO and ²H-lactate production were both much lower in AML12 cells.

TABLE 1

| | HUH-7 | | AML12 | |
|---|---|---|---|---|
| | | number of | | number of |
| Cell lines | μmol/L | μmol of ²H | μmol/L | μmol of ²H |
| Consumed | 3650.0 | 25550.0 | 586.52 | 4105.60 |
| [²H₇]glucose | | | | |
| Lactate-CHD₂ | 2442.47 | 4884.9 | 353.07 | 706.14 |
| Lactate-CH₂D | 1520.0 | 1520.0 | 229.26 | 229.26 |
| HDO | 18240.0 | 18240.0 | 2003.12 | 2003.12 |
| Number of μmol | | 24644.9 | | 2938.52 |
| of ²H in ²H- | | | | |
| lactate & HDO | | | | |

Warburg metabolism is phenotypic of many cancers. Increased lactate production is a consequence of increased glucose consumption and reduced pyruvate dehydrogenase flux in cancer cells. This study has successfully demonstrated the metabolic conversion of [²H₇]glucose to lactate isotopomers and also quantitatively accounted for HDO production in these models. Conversion of DHAP and GA3P into lactate—CH₂D and lactate-CHD₂ can be measured efficiently by this method as well, which also indicates the ²H loss from [²H₇]glucose during glycolysis. Quantitation of HDO production may provide a simplified means for metabolic imaging of the deuterium signal specific to cancer cell glucose uptake, analogous to [¹⁸F]-FDG PET, with superior signal-to-noise of other metabolite signals, as well as ²H MRS imaging.

Based on parameters for oral (Gunning, R. R. Jama 240:1611 (1978)) and intravenous administration of glucose in humans and estimates of tumor geometry and cellularity (Kondo, F. Hepatology International 3:283-293 (2009)), HDO enrichment in the peritumoral area may reach several percent, sufficient to observe lengthening of ¹H T₁ due to less efficient dipole-dipole relaxation (Sherry, A. D., et al. Analytical Biochemistry 52:415-420 (1973)). T₁ weighted ¹H imaging might therefore also be diagnostic, which would remove the need for purpose built ²H detection coils. A useful feature of our proposed tracer method versus methods based on TCA intermediate/lactate ratios is that it obviates the need to correct for ²H loss between the two species, reducing inter-subject variance and providing for easier extension to multiple tumor types.

From a practical point of view, {²H}¹H—NMR experiments are easy to set up and require minimal time compared to direct ²H detection for ²H-lactate. Measuring the kinetics of the lactate isotopomer production from deuterated glucose by utilizing the more sensitive ¹H—NMR experiment makes an easy method for measuring glycolytic rates. It is to be determined if this method could be translated for in vivo use. Lactate detection by ¹H MRS is typically confounded by overlap of resonances derived from FAs. With a large FA signal the difference spectrum approach used here would have to be carefully implemented to prevent improper subtraction of the FA peaks and uncertainty in quantification of the ²H lactate. As compared to ¹³C based editing sequences, this method does not have to contend with a natural abundance correction. Also, the isotope shifts could potentially provide a more robust subtraction signal. The differential T₁ of the lactate isotopomers is more difficult to take advantage of, as the T₁ is lengthened upon ²H-enrichment. Standard T₁ weighting would not provide contrast from the isotopomers. Specific choice of the delay time might allow the lactate—CH₃ signal to be nulled which the ²H isotopomers remain using inversion recovery schemes.

In conclusion, this study demonstrates that administration of [²H₇]glucose can trace Warburg metabolism in cancer cells using two readily applicable isotopomer detection methods: evolution of HDO, detected by ²H—MR, and of three different isotopomers of lactate, detected by {²H}¹H—NMR with difference spectroscopy to remove interference from fatty acids. As compared to earlier results with [6,6-²H₂]glucose, this method retains the selectivity of lactate enrichment, but with the increased likelihood of HDO generation, amplifies the sensitivity of the experiment to increased glycolytic flux. Either technique can be implemented on preclinical or clinical hardware and may provide the basis for a safe, zero-dose method of imaging cancer glucose uptake and Warburg metabolism.

### Example 2: HDO Imaging of the Rat Brain Following Metabolism of [²H₇]Glucose

Introduction

Glucose is the principal source of energy in the brain, and its dynamic uptake and utilization remain topical after decades of research. Metabolic imaging using MR based methods has traditionally been limited to ¹H based spectroscopic detection of metabolites via chemical shift imaging (CSI) or single voxel spectroscopy (Frahm J, et al. Journal of Magnetic Resonance (1969) 1985 64:81-93; Bottomley P A. Ann N Y Acad Sci 1987 508:333-348). While these methods have been of immense use in clinical research, sensitivity issues have made their translation to the clinic problematic. More recently, hyperpolarized (HP)¹³C imaging has been used to study heart, liver, and brain metabolism, as well as changes in metabolism associated with various cancers (Merritt M E, et al. Proceedings of the National Academy of Sciences 2007 104:19773-19777; Salamanca-Cardona L, et al. Cancer Metab 2015 3:9). HP imaging uses the large gain in signal-to-noise ratio (SNR) made possible by dynamic nuclear polarization (DNP) to produce chemically selective images that reflect changes in metabolic flux (Comment A, et al. Biochemistry 2014 53:7333-7357). However, DNP is limited to molecules having long relaxation times amenable to sample transfer from the polarizer to the patient. Many important metabolites such as glucose have short relaxation times, making clinical translation more challenging (Kurhanewicz J, et al. Neoplasia 2019 21:1-16). 2-[¹⁸F]-fluorodeoxyglucose positron emission tomography (FDG-PET) is widely utilized for cancer diagnosis, staging, and treatment assessment (Kelloff G J. Clinical Cancer Research 2005 11:2785-2808). However, the specificity of FDG-PET is limited in the brain, where normally high glucose uptake and non-cancerous inflammation can give false positive results (Shreve P D, et al. Radiographics 1999 19:61-77). Additionally, FDG-PET is limited to glucose uptake and phosphorylation; glycolytic and TCA cycle metabolism are invisible to this technique.

Another approach that utilizes the chemical selectivity of MR utilizes ²H labelled substrates to image metabolic turnover. In vivo deuterium magnetic resonance spectroscopy (DMRS) has been demonstrated using [6,6-²H₂]glucose to measure glucose uptake and glutamate/glutamine (glx) production over time, providing an alternative method for turnover studies in the brain (Lu M, et al. J Cereb Blood Flow Metab 2017 37:3518-3530). The same glucose isotopomer was used to image human glioblastoma (De Feyter H M, et al. Sci. Adv. 2018 4:eaat7314). However, the deuterated glucose isotopomer chosen for the study predetermined that downstream deuteration at lactate-C3 and glx-C4 would necessarily be the metabolic readouts of glucose metabolism. These peaks are slowly enriched as a function of time, and are most easily imaged using the CSI technique, as they are smaller peaks relative to the deuterated water (HDO) signal that arises both from natural abundance contributions and from a limited set of exchange reactions that eliminate the ²H label from the methyl position of lactate. As disclosed herein, [²H₇]glucose is an alternative agent, and HDO evolution accurately reflects total glucose uptake (Mahar R, et al. Sci Rep 2020 10:8885), in analogy to FDG-PET (Vlassenko A G, et al. Neurolmage 2006 33:1036-1041), but with detection of glucose downstream metabolism and without the use of ionizing radiation.

This example demonstrates the application of this method in vivo to image glucose consumption in the rat brain. Administration of [²H₇]glucose via the tail vein produced deuterated lactate and glx analogous to earlier reports, but shown here for the first time is that HDO generated from metabolism can be imaged in the rodent brain using gradient echo based methods as opposed to CSI. Using methods other than CSI to detect metabolic flux fundamentally transforms the technique, allowing excellent in plane resolution and faster acquisition times. By using HDO evolution as a metric of glucose utilization in the brain, a biomarker of cerebral glycolysis and oxidative flux is introduced that is imaged with superior sensitivity and resolution compared to previous approaches.

Methods

Chemicals and Tracer

[1,2,3,4,5,6,6-²H₇]-D-glucose (or [²H₇]glucose) was purchased from Sigma Aldrich, USA. Isoflurane anesthesia was purchased from Aspen Veterinary Resources, M O, USA. Sterile filtered saline solution was prepared to dissolve the [²H₇]glucose solution. Heparin at 1000 USP/ml of glucose solution was also utilized to stop the clotting of blood during animal preparation.

Animal Experimental Setup

All experimental procedures were approved by the University of Florida Institutional Animal Care and Use Committee. Female Sprague Dawley rats (body weight: ˜220 g) were purchased from Jackson Laboratories and acclimatized in the University's animal care facilities. All rats were in the fed state. The rat was anesthetized with 2% isoflurane in a mixture of O₂ and N₂ gases. The tail vein was catheterized using a 23-gauge needle for the infusion of [²H₇]glucose tracer. The body temperature of the rat was maintained at 37° C. using a heated circulating water pad. The respiratory rate was monitored during the entire period of the experiment. After shimming the magnet, three baseline ²H spectra were acquired. Subsequently, a 2 minute tail vain infusion of [²H₇]glucose (1.95 g/kg body weight and dissolved in 2.3 mL saline) was administered. Blood was drawn after the end of spectroscopy and imaging experiments via saphenous vein and blood plasma was separated by centrifugation of the blood at 4° C. for 10 minutes. Circulating blood glucose was assessed by a blood draw and glucometer (Evancare, Medline Industries, Northfield, IL) prior to infusion and 3 hours after the glucose injection.

MRI System

Animal experiments were performed on an 11.1 T Magnex magnet (Magnex Scientific Ltd.) interfaced to a BrukerAvance Ill HD console running on ParaVision 6.0.1 (Bruker Instruments, Billerica, Mass.). The MRI system was equipped with RRI BFG-240/120-S6 gradient coil with the bore size of 120 mm and capable of producing 100 mT/m with 200 μs rise time. A shim map was generated for localized shimming on rat brain using an 85 mm home-built, actively-decoupled, linear volume ¹H transmit-receive coil. The coil was tuned to the ¹H NMR frequency at 470 MHz. A home-built 14 mm diameter, circular surface coil tuned to 72.26 MHz was used to acquire ²H spectra and images.

Deuterium Magnetic Resonance Spectroscopy

The ²H coil was placed over the rat head and a 3D printed phantom containing approximately 200 μl of natural abundance water was placed on the top of the coil to confirm positioning. The ²H RF pulse and flip angle were optimized to maximize the ²H signals from the rat brain. A partial signal contribution from the natural abundance ²H of the water phantom was unavoidable but was accounted for by subtraction of the signal areas from runs that followed the glucose injection. A single pulse acquire sequence was used to obtain ²H NMR spectra, with an observed linewidth of approximately 40 Hz. Spectra were acquired with a 1 kHz width and 256 data points for each free induction decay (FID), a repetition time of 300 ms, and a flip angle of 60° with 256 averages. The total acquisition time for each spectrum was 1.28 minutes. A total of 30 spectra were acquired for a 38-minute time period. Two additional ²H NMR spectra from the rat brain were acquired at 2 hours and 2.5 hours following [²H₇]glucose infusion. Pyrazine-D₄ (2.5 mM, internal standard) was added in each plasma sample to calculate the HDO concentration in plasma. ²H NMR spectra were collected on Bruker 14 T NMR magnet system equipped with a 1.7 mm TCl CryoProbe and Avance Neo console (Bruker BioSpin). ²H NMR spectra of plasma samples were acquired at 92.13 MHz resonant frequency with an acquisition time of 1.0 s, relaxation delay of 2 s, and 3072 scans.

Deuterium Magnetic Resonance Imaging

Axial ¹H images of rat brain were acquired using a Bruker Fast Low Angle Shot (FLASH) gradient echo sequence. The two-dimensional (2D) acquisition matrix size of 128×128, field-of-view (FOV) of 50×50 mm², and 8 averages were used to acquire 10 slices of rat brain with 2 mm thickness. Before [²H₇]glucose infusion, a natural abundance deuterium ²H image of the rat brain was acquired using the FLASH sequence, with the ²H water signal on resonance. Two deuterium images were also acquired at 38 minutes and ˜2 hours post-[²H₇]glucose infusion. The following parameters were used to acquire an axial ²H images of the rat brain: 2D acquisition matrix size 32×32, FOV of 50×50 mm², slice thickness of 14 mm, repetition time of 100 ms, 256 signal averages, 1.416 ms echo time (TE), and 34.2° flip angle with an in-plane resolution of 1.56 mm². Each image took 13 minutes to acquire. The Bruker multi-gradient echo (MGE) imaging experiment was utilized to acquire the echo images for separating HDO-only and [²H₇]glucose-only images in the rat brain during the pseudo-steady state. Axial two-echo images were acquired with echo times of 11.1 ms (GE1) and 16.6 ms (GE2), acquisition matrix size of 32×32, FOV of 40×40 mm 2, and slice thickness of 14 mm. The in-plane resolution was 1.25 mm². Proton intensity images of the rat brain was also acquired with the same FOV as deuterium images for co-registration purposes.

NMR and MRI Data Processing

²H NMR data analysis was performed using MestReNova v14.0.1-23284 (Mestrelab Research S. L., Santiago, Spain). For processing of the ²H NMR, each spectrum was Fourier transformed with an exponential window function of 2.0 Hz, zero-filling of 512 data points and automatic phase correction was also applied. Peak areas were extracted for HDO, [²H₇]glucose including the anomeric signal, deuterated glx (glutamate/glutamine) and lactate from the ²H NMR spectra of the rat brain. 2D ¹H and ²H images were processed in the ImageJ software (NIH, Bethesda, Md.). Since the acquired ²H image size was 32×32 and the ¹H brain image was 128×128, the deuterium image was scaled using bicubic interpolation to 128×128 for co-registration and overlay of the images. This resulted in a final, interpolated, in plane resolution of 0.39 mm². MGE echo images were reconstructed to 128×128 matrix size with gaussian windowing filter using Bruker ParaVision 6.0.1 software. Reconstructed echo images were imported to MATLAB for further processing to separate the contribution of HDO and [²H₇]glucose to the rat brain DMRI. The Dixon reconstruction algorithm (Ragan D K, et al. J. Magn. Reson. Imaging 2010 31:510-514) was applied to reconstruct the HDO-only and [²H₇]glucose-only images from the MGE images. T₂ relaxation times of HDO and [²H₇]glucose were also used as part of the Dixon reconstruction (De Feyter H M, et al. Sci Adv 2018 4:eaat7314).

Results

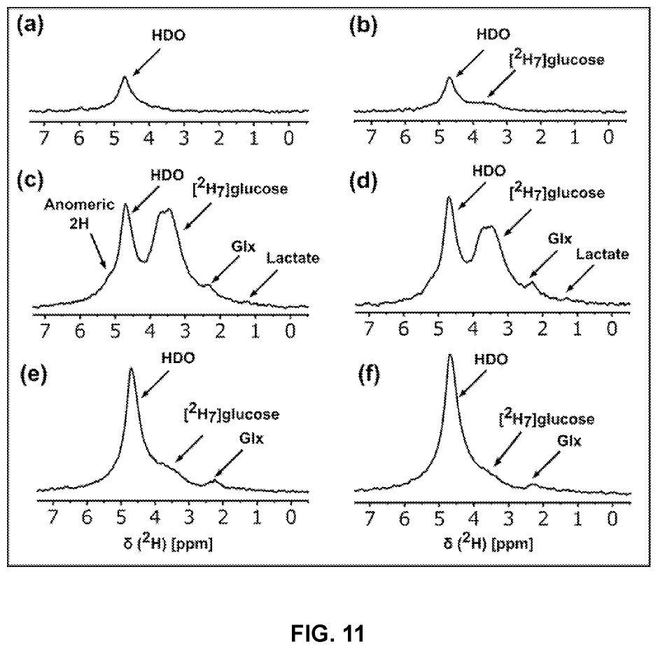

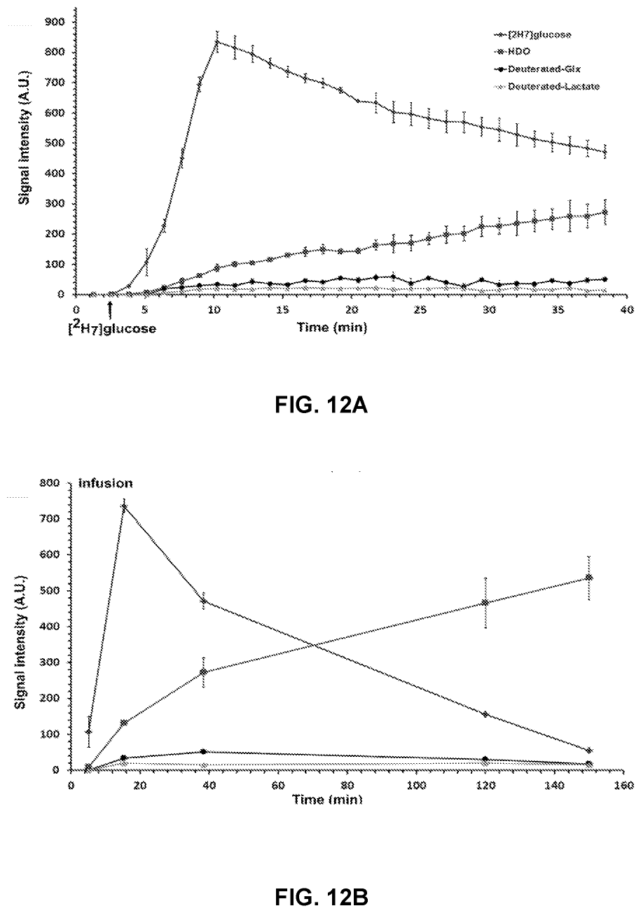

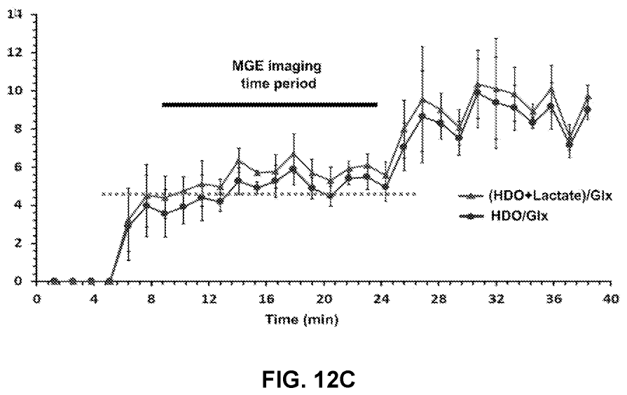

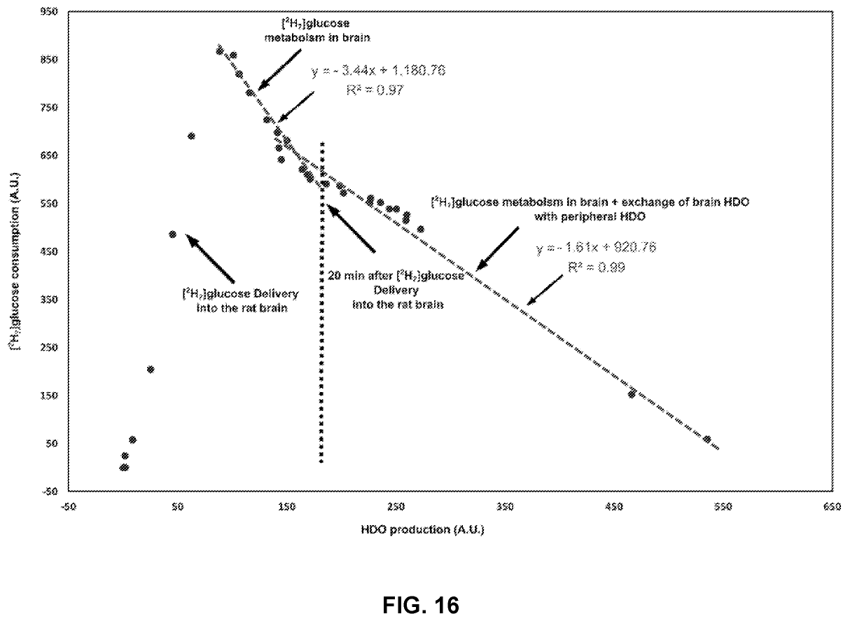

The initial blood glucose was measured as 8.0±1.4 mM (n=4) prior to infusion and the final blood glucose concentration at 3 hours after injection was 7.1±1.8 mM (n=4). The natural abundance HDO water signal (FIG. 11A) obtained using a surface coil with the 200 μl phantom in place is assumed to reflect the 0.015% enrichment of HDO in water (approximately 13.2 mM, assuming the brain is 75% water by mass, and accounting for the phantom). The subsequent panels show the arrival of a bolus of [²H₇]glucose and its metabolism to glx and lactate (FIG. 11C, 11D). Over the course of 2.5 hours, the glucose bolus is almost completely metabolized, returning to a level just above the detection limit (FIG. 11F). Most importantly, the HDO signal increased rapidly initially, then continued to rise at a slower rate as metabolism continues (FIG. 12). At the end of the experiment, the HDO signal dominates both the residual glucose and the remaining glx signal (FIG. 11F). The kinetics of the deuterated precursor and products includes the HDO signal after a correction for the natural abundance HDO (FIG. 12). The lactate and glx signals display similar kinetics, rising with increasing glucose concentration, then slowly decreasing, albeit at a slower rate than the glucose signal. It should be noted that turnover of the lactate and glx pools could continue to contribute to the HDO pool size. At longer time points (FIG. 12B), the HDO signal does not fall, but continues to increase to a final estimated concentration approximately 3.5×greater than the natural abundance signal, corresponding to approximately 48±10 mM (N=3). A plot of the HDO signal intensity versus the glucose consumption showed a bimodal response following the initial arrival of the glucose tracer (FIG. 16). The glx intensities were plotted against HDO and HDO plus lactate intensities as a means of assessing the correlation between HDO production, LDH flux, and TCA cycle activity (FIG. 12C). After the initial appearance of the metabolites, a pseudo-steady state for the ratios is reached in the range of 10-24 minutes.

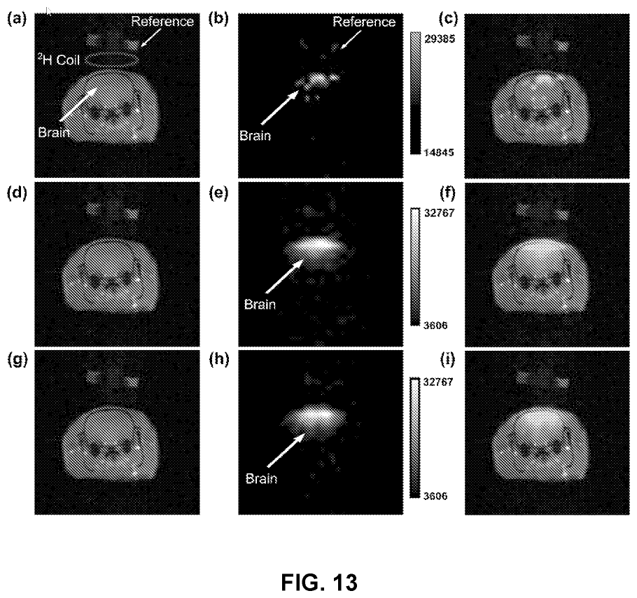

Two gradient echo images were acquired at approximately 38 minutes and 2 hours post infusion. The initial image prior to injection shows a very faint background HDO signal, which increases significantly with time (FIG. 13F, 13I). The improved SNR allows a rudimentary coil sensitivity profile to be visualized. The image of FIG. 13E was acquired with HDO on resonance beginning at 38 minutes post infusion, but it is subject to a significant chemical shift artifact associated with the glucose signal. At 2.5 hours after infusion the HDO is by far the largest signal, and does not have a significant contribution from the glucose signal.

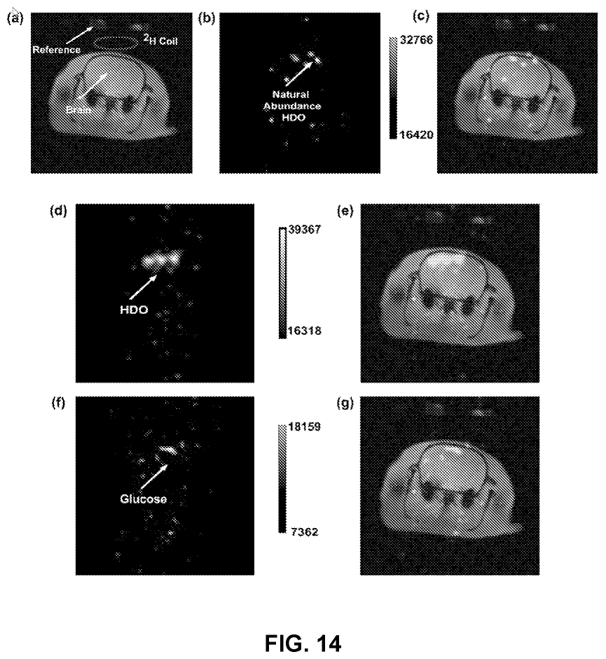

To distinguish the HDO signal from the [²H₇]glucose substrate, an MGE based two-point Dixon method was implemented during the initial time period (approximately 10-24 minutes) of the post-[²H₇]glucose infusion and metabolism (FIG. 14). FIGS. 14D and 14F are the HDO-only and [²H₇]glucose-only images, respectively. Blood plasma collected at the end of the experiment was used to assess the final body water HDO enrichment, which was determined to be 86.9±5.16 mM (n=4).

Discussion

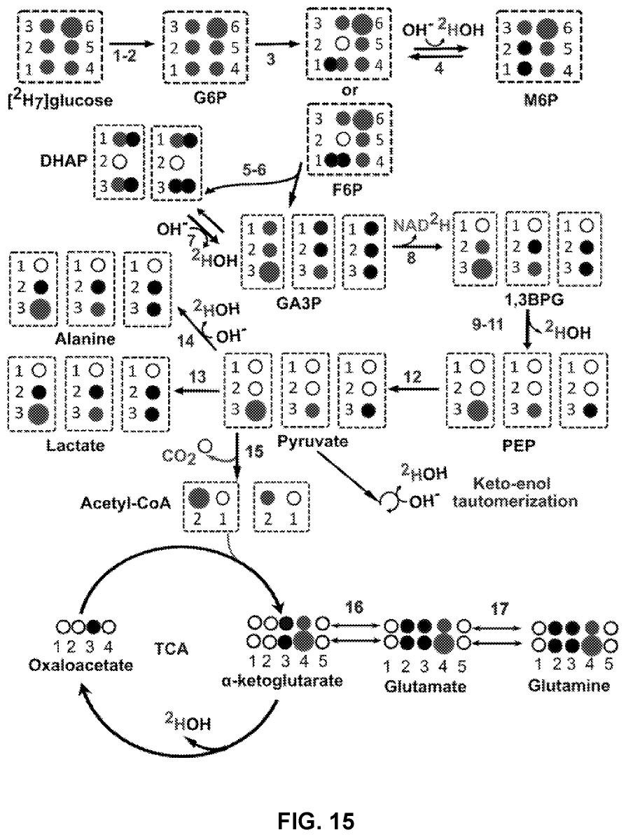

The brain image of FIG. 13 is the first demonstration of the efficacious use of HDO generated from [²H₇]glucose as a means for producing metabolically sensitive MR images using simple gradient echo methods (Kreis F, et al. Radiology 2020 294:289-296). By choosing the metabolic product HDO as the target, drawbacks associated with imaging relatively low concentration metabolites, like lactate and glx, are bypassed. Metabolism of [²H₇]glucose can produce HDO via flux into the 3-carbon glycolytic intermediates, and via oxidation of the downstream deuterated acetyl-CoA (FIG. 15). It can also be produced by multiple enzyme catalyzed exchange reactions like triose phosphate isomerase (Browning J D, et al. American Journal of Physiology-Endocrinology and Metabolism 2012 303:E1304-E1312; Leadlay P F, et al. Biochemistry 1976 15:5617-5620), and alanine aminotransferase (Funk A M, et al. Journal of Magnetic Resonance 2019 301:102-108), etc. HDO production from the C1/C2 positions of fructose-6-phosphate and mannose-6-phosphate can also occur via phosphomannoseisomerase (Chandramouli V, et al. American Journal of Physiology-Endocrinology and Metabolism 1999 277:E717-E723). In this case, the possible action of the pentose phosphate pathway was neglected. Of the possible HDO generating processes, only the keto-enol tautomerization (Ben-Yoseph O, et al. Magn. Reson. Med. 1994 32:405-409) is purely chemical in nature. Previous work suggests that it could account for a maximum of 8% of the HDO generation when using [6,6-²H₂]glucose (De Feyter H M, et al. Sci. Adv. 2018 4:eaat7314), indicating that its contribution in this case should be very small in relative terms. In recent work in Huh-7 cancer cells incubated with [²H₇]glucose, unlabeled lactate increased with time, suggesting significant deuterium loss during glycolysis (Mahar R, et al. Sci Rep 2020 10:8885). The stoichiometry of the metabolism of the deuterated tracer is such that the number of HDO molecules produced by glycolysis versus those deposited into glx is either 6 to 1 or 5 to 2, depending upon whether the pyruvate produced by glycolysis is either doubly or singly deuterated (FIG. 15). Multiple reactions, including equilibration at M6P, alanine, or pyruvate keto-enol tautomerization can result in a singly deuterated pyruvate entering the TCA cycle. The average stoichiometric ratio of HDO produced to the deuterons of the C4 position of glx is therefore 4.25. Note that due to the action of isocitrate dehydrogenase, deuterium should be totally eliminated from TCA cycle intermediates on the 2^{nd} turn of the cycle, implying that glx enrichment is due to “first pass” kinetics only. Therefore, the 4.25 value should be robustly conserved in normal tissue. This value is illustrated as the dotted line in FIG. 12C, and the experimentally determined ratio closely conforms to this value for approximately 15 minutes after the maximal circulating glucose concentration in the brain is achieved (N=3). Accounting for deuterium deposited into lactate and HDO as compared to glx slightly increases the value, and would probably be a more accurate metric of glycolytic flux if an absolute rate of incorporation were to be determined using kinetic modeling (FIG. 12C). After this time period, HDO continues to rise more rapidly than lactate and glx. The increasing HDO signal in the brain late after injection was attributed to the influx of HDO generated by glucose metabolism in peripheral tissues. This data strongly suggests that imaging HDO during the time-period of 10-24 minutes is equivalent to separate estimates of lactate and glx kinetics, albeit with significantly higher sensitivity. This time period was used when the HDO/glucose two-point Dixon imaging was applied (FIG. 14).

As detailed in the results, the first ²H image after glucose injection (FIG. 13E) is a convolution of the HDO and glucose signals. These images were of sufficient SNR to allow us to implement frequency selective imaging. A multi-gradient echo two-point Dixon method was optimized to produce HDO versus glucose images (FIGS. 14D and 14F) in analogy to fat-water imaging (Ma J. J. Magn. Reson. Imaging 2008 28:543-558). The in-plane resolution was reduced to 1.25 mm² in these images, based only on the imaging parameters prior to data processing. CSI could not produce images of this resolution even at time periods approximately 4 times as long for acquisition. Increasing imaging specificity could be encoded using multi-point Dixon methods to account for glx and lactate signals (Wang Y, et al. J Magn Reson Imaging 1998 8:703-710), but this approach was not implemented for this study. The maximal glucose intensity was treated at approximately 3.4 ppm as the center of the glucose signal. This is obviously a compromise to maintain the simplicity of the imaging method. Phase dispersion associated with the inhomogeneously broadened glucose signal likely costs sensitivity in the reconstructed glucose image of FIG. 14F. it is believed that the HDO signal is largely free from these issues, though a small component originating from the anomeric deuterium of glucose is also present under the HDO signal. In total, the HDO image should serve as an accurate biomarker of glycolytic flux. The HDO image should also recapitulate glx kinetics in the case of normally functioning brain tissue. The HDO image would fail to report upon TCA cycle kinetics in the case of a Warburg phenotype.

While the images here were acquired with a significant slice thickness that must cause significant partial volume effects, the overall SNR indicates that thinner slices will be readily obtainable once the protocol and hardware are further optimized. The fact that the HDO concentration in the brain remains below that of body water enrichment at 3 hours indicates that influx of HDO produced outside the brain is not as rapid as might be expected. This is confirmed by the HDO appearance at the longer time points (FIG. 12), which does not appear to be approaching a limit even at 3 hours. Additionally, the perdeuterated glucose will not be fully consumed to produce HDO, because glucose is readily distributed throughout the body. For example, in the liver, it could be stored in the form of glycogen (De Feyter H M, et al. Sci Adv 2018 4:eaat7314), or it could participate in lipogenesis. Both of these processes would limit the total amount of HDO eliminated from [²H₇]glucose given by IV injection. A significant consideration for this method is the physiological effect of administering what is equivalent to a glucose tolerance test to the subject (Riis-Vestergaard M J, et al. Int J Obes 2020 44:1417-1427). Insulin sensitivity might play a large role in the interpretation of metabolic images produced by this method.

Metabolic imaging of the brain has been a research target driven by both pathophysiological considerations, like stroke and cancer, and by studies of brain structure and cognition as a basic science. Glucose is the primary source of energy in the brain, but the dynamic response of glucose utilization remains topical. In cancer, aerobic glycolysis is a common metabolic phenotype of many brain malignancies. This change in metabolic programming, often termed Warburg metabolism (Warburg O. Science 1956 123:309-314), is effectively targeted using FDG-PET, which measures excessive glucose uptake but does not otherwise interrogate downstream glucose metabolism. FDG-PET has been used to study glucose metabolism in the brain for forty years (Phelps M E, et al. Ann Neurol. 1979 6:371-388; Villien M, et al. Neurolmage 2014 100:192-199), and has been used to measure functional responses to stimuli (Vlassenko A G, et al. Neurolmage 2006 33:1036-1041). However, total radiation burden makes PET unsuitable in pediatric populations and prevents its serial use in patients (Chawla S C, et al. Pediatr Radiol 2010 40:681-686). In cognitive psychology, tremendous effort has been expended to map the effects of brain activation using the BOLD methodology (fMRI) (Yamada H, t al. NeuroReport 1997 8:3775-3778; Ugurbil K, et al. Annu. Rev. Biomed. Eng. 2000 2:633-660). Similar techniques measuring cerebral metabolic rate of O₂ consumption (CMRO2) have been used to estimate glucose oxidation as well (Rodgers Z B, et al. J. Cereb. Blood Flow Metab. 2016 36:1165-1185). All these methods indirectly infer glucose metabolism without measuring the downstream metabolites. Hyperpolarized ¹³C does offer the ability to directly detect lactate concentrations and lactate dehydrogenase flux when using HP pyruvate as the tracer (Kurhanewicz J, et al. Neoplasia 2019 21:1-16; Merritt M E, et al. Proceedings of the National Academy of Sciences 2007 104:19773-19777), but this technique demands a special pre-polarizer to produce the imaging agent. Furthermore, hyperpolarized glucose is obviously an analogous method, but the short carbon-13 T₁ of glucose makes it difficult to translate to human studies (Kurhanewicz J, et al. Neoplasia 2019 21:1-16). In contrast, imaging of deuterated agents requires only the addition of a detection coil tuned to the appropriate frequency, and therefore could be easily disseminated to clinical sites. As shown in this Example, by imaging HDO, an end-product of [²H₇]glucose metabolism, one can produce an image of glucose disposal. This modality should be complimentary to ¹H based methods, and kinetic analysis should yield absolute rates of glycolysis, and TCA cycle turnover in healthy tissues.

### CONCLUSION

Metabolic imaging of HDO can therefore be used as a biomarker of [²H₇]glucose, lactate, and glx metabolism. Spectrally selective imaging of HDO and glucose using a two-point Dixon method was accomplished with a simple multi-gradient echo sequence. This modality facilitates higher in-plane resolution and faster acquisition of imaging data than possible with CSI methods.

### Example 3: Imaging the NAFLD to NASH Transition with Metabolically Sensitive ²H MRI

Nonalcoholic steatohepatitis (NASH) affects about one-third of all patients with obesity and type 2 diabetes mellitus (T2DM) becoming a major risk factor for the development of cirrhosis and hepatocellular carcinoma (HCC) (Anstee, Q M, et al. Nature reviews Gastroenterology & hepatology 2019 16(7):411-428). NASH is the second leading cause of liver transplantation in the USA. The transition from nonalcoholic fatty liver disease (NAFLD) (considered a rather “benign” condition that affects 70% of obese insulin-resistant patients with T2DM, not leading to cirrhosis) to NASH (a stage of hepatocyte necrosis, inflammation, and often active fibrogenesis leading to cirrhosis) which involves induction of abnormal fatty acid p-oxidation, leading to chronic inflammation and liver fibrosis (Younossi, Z, et al. Nature reviews Gastroenterology & Hepatology 2018 15(1):11). Current plasma biomarkers or metabolic/other imaging techniques are needed that can identify NAFLD and end-stage disease (i.e., advanced fibrosis or cirrhosis) but not early disease (i.e., NASH) when the disease is at a stage that demands diagnosis and therapeutic intervention. Therefore, staging of the disease is central to the effective treatment.

NAFLD is becoming a nearly ubiquitous consequence of the western diet, with the most recent estimates suggesting a prevalence as high as 55-65% in patients with obesity or T2DM in the United States. As the disease progresses, NASH develops by mechanisms that remain unclear in about one-third of patients and in many it results in cirrhosis. Within the next 5 years, NASH will become the number one cause of liver transplant in the United States. NASH not only can lead to cirrhosis, but is also associated with a higher incidence of hepatocellular carcinoma (HCC). There has been tremendous effort expended on identification of biomarkers and imaging (FibroScan or MR elastography) for the diagnosis of fibrosis in patients with NASH, but both of these modalities are insensitive to fatty acid oxidation (increased or decreased) and inflammatory response, two primary characteristics of NASH onset. Ideally, one would like to make the diagnosis of NASH before the development of liver fibrosis but the available biomarkers and imaging have not been good enough for widespread use. The only definitive method for the diagnosis of NASH is liver biopsy, which is expensive, sampling dependent, poorly accepted by patients and clinicians, and in a small percentage associated with complications, rarely even death. Development of an imaging method which could directly assess global hepatic metabolism and monitor patients for the NAFLD to NASH transition would transform our ability to manage this increasing prevalent, costly disease.

In mice, the transition from NAFLD to NASH is accompanied by an upregulation of fatty acid (FA) oxidation in the tricarboxylic acid (TCA) cycle, in seeming contradiction to the visible deposition of fat in the liver. Upregulated fatty acid oxidation in NASH has also been implicated in human studies. Increased FA oxidation leads to the accumulation of lipotoxic intermediates as well as increased oxidative stress in the liver. Previously carbon-13 magnetic resonance (MR) has been used to interrogate TCA cycle turnover, but only at tremendous expense (˜$25,000/patient in [1-¹³C]acetate label alone). Recently, it was demonstrated that metabolic imaging with deuterated glucose and acetate, and ²H MRI is feasible in human brain (De Feyter, et.al., Science, 2018). The low sensitivity of the ²H nucleus can be overcome by fast repetition of the experiment, as the MR T₁ of deuterium is very short. Octanoate is a medium chain fatty acid that does not use CPT-1 for transport into the mitochondria. Therefore it is avidly and rapidly p-oxidized by the liver. Therefore, ²H MRI in patients after a bolus of perdeuterated octanoate can produce a metabolically sensitive image suitable for identifying the NAFLD to NASH transition.

A robust imaging method for detecting NASH development could transform the field and radically modify current patient management. Early intervention in patients with NASH, has been shown to prevent or reverse hepatocyte necrosis and liver inflammation/fibrosis (lifestyle targeted to high-risk patients, bariatric surgery vitamin E and pioglitazone; AASLD 2018 guidelines). Early and accurate staging can allow the physician to determine the moment when patient care should be modified, reducing costs, and improving patient outcomes.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for imaging a tissue in a subject, comprising
(a) administering to the subject a composition comprising deuterium-labeled glycolytic substrate, a deuterium-labeled fatty acid substrate, or a combination thereof; and
(b) imaging the subject with deuterium magnetic resonance imaging (DMI) to detect hydrogen-deuterium oxide (HDO) in tissues of the subject,
wherein detection of HDO in a location within the tissue of the subject is an indication of glucose utilization and/or fatty acid oxidation in the tissue of the subject.

2. The method of claim 1, wherein detection of elevated glucose utilization in the tissue is an indication of a cancer.

3. The method of claim 1, wherein the tissue is selected from the group consisting of brain, heart, muscle, skin, liver, pancreas, spleen, kidney, stomach, gut, and lungs.

4. The method of claim 3, wherein detection of impaired glucose utilization and/or fatty acid oxidation is an indication of injury or pathology.

5. The method of claim 3, wherein detection of elevated glucose utilization and/or fatty acid oxidation is an indication of metabolic activation.

6. The method of claim 1, wherein the deuterium-labeled glycolytic substrate comprises deuterated glucose.

7. The method of claim 6, wherein the deuterated glucose comprises at least 3 deuterium atoms.

8. The method of claim 7, wherein the deuterated glucose comprises [²H₇]glucose.

9. The method of claim 1, wherein the deuterium-labeled fatty acid substrate comprises deuterated octanoate.

10. The method of claim 9, wherein the deuterated fatty acid comprises at least 3, deuterium atoms.

11. The method of claim 9, wherein the deuterated fatty acid is perdeuterated.

12. The method of claim 1, wherein the DMI comprises ²H—NMR imaging.

13. The method of claim 12, wherein the DMI further comprises ¹H—NMR imaging that uses T₁ or T₂ weighting.

14. The method of claim 1, wherein the DMI is further used to detect deuterated glucose uptake in the tissue of the subject.

15. The method of claim 1, wherein the DMI is further used to detect deuterated lactate production in the tissue of the subject.

16. The method of claim 1, wherein the DMI is further used to detect deuterated glutamate/glutamine (glx) production in the tissue of the subject.

17. The method of claim 1, wherein the DMI is further used to detect a ratio of deuterated lactate/glx.

18. The method of claim 1, wherein the DMI is further used to detect an increased HDO signal as a biomarker of metabolic flux.

19. The method of claim 1, wherein the DMI is further used to detect any combination of ratios or HDO to lactate, or HDO to glx.

20. The method of claim 1, wherein imaging step (b) occurs within 8 to 24 minutes of step (a).

21. The method of claim 20, further comprising repeating step (b) at additional time points to determine a rate of HDO enrichment in the tissue of the subject.
